SEQUENCE LISTING (4 sequences)

SEQ ID NO: 1 - protein, 108 aa, homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 2 - protein, 118 aa, homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 3 - protein, 9 aa, homo sapiens
IMDQVPFSV

SEQ ID NO: 4 - protein, 9 aa, homo sapiens
YLEPGPVTV

(19) United States
(12) Patent Application Publication
(10) Pub. No.: US 2013/0064831 A1
(43) Pub. Date: Mar. 14, 2013

(54) IMMUNOTHERAPEUTIC DOSING REGIMENS AND COMBINATIONS THEREOF

(71) Applicant: Rachel Wallach Humphrey, Princeton, NJ (US)

(72) Inventor: Rachel Wallach Humphrey, Princeton, NJ (US)

(73) Assignee: Bristol-Myers Squibb Company

(21) Appl. No.: 13/698,300

(22) PCT Filed: May 16, 2011
(86) PCT No.: PCT/US11/36626

§ 371 Date: Nov. 16, 2012

Related U.S. Application Data
(60) Provisional application No. 61/345,334, filed on May 17, 2010.
(60) Provisional application No. 61/452,841, filed on Mar. 15, 2011.

(51) Int. Cl. A61K 39/395; A61P 37/02; A61P 35/00

(52) U.S. Cl. 424/142.1; 424/173.1

(57) ABSTRACT

The invention described herein relates to therapeutic dosing regimens and combinations thereof for use in enhancing the therapeutic efficacy of immunotherapeutic agents e.g. CTLA-4 antagonists such as Ipilimumab or Tremelimumab in combination with one or more chemotherapeutic agents in cancer patients.

This application claims benefit to provisional application U.S. Ser. No. 61/345,334 filed May 17, 2010; and to provisional application U.S. Ser. No. 61/452,841, filed Mar. 15, 2011; under 35 U.S.C. §119(e). The entire teachings of the referenced applications are incorporated herein by reference.

### FIELD OF THE INVENTION

The invention described herein relates to therapeutic dosing regimens and combinations thereof for use in enhancing the therapeutic efficacy of immunotherapeutic agents in combination with one or more chemotherapeutic agents.

### BACKGROUND OF THE INVENTION

The National Cancer Institute has estimated that in the United States alone, 1 in 3 people will be struck with cancer during their lifetime. Moreover, approximately 50% to 60% of people contracting cancer will eventually succumb to the disease. The widespread occurrence of this disease underscores the need for improved anticancer regimens for the treatment of malignancy.

Due to the wide variety of cancers presently observed, numerous anticancer agents have been developed to destroy cancer within the body. These compounds are administered to cancer patients with the objective of destroying or otherwise inhibiting the growth of malignant cells while leaving normal, healthy cells undisturbed. Anticancer agents have been classified based upon their mechanism of action, and are often referred to as chemotherapeutics. The combination of chemotherapeutics with immune modulating agents has been gaining increasing acceptance in the oncology field.

The vertebrate immune system requires multiple signals to achieve optimal immune activation; see, e.g., Janeway, Cold Spring Harbor Symp. Quant. Biol., 54:1-14 (1989); Paul, W. E., ed., Fundamental Immunology, 4th Ed., Raven Press, N.Y. (1998), particularly Chapters 12 and 13, pp. 411-478. Interactions between T lymphocytes (T cells) and antigen presenting cells (APC's) are essential to the immune response. Levels of many cohesive molecules found on T cells and APC's increase during an immune response (Springer et al., Ann. Rev. Immunol., 5:223-252 (1987); Shaw et al., Curr. Opin. Immunol., 1:92-97 (1988)); and Hemler, Immunology Today, 9:109-113 (1988)). Increased levels of these molecules may help explain why activated APC's are more effective at stimulating antigen-specific T cell proliferation than are resting APC's (Kaiuchi et al., J. Immunol., 131:109-114 (1983); Kreiger et al., J. Immunol., 135:2937-2945 (1985); McKenzie, J. Immunol., 141:2907-2911 (1988); and Hawrylowicz et al., J. Immunol., 141:4083-4088 (1988)).

T cell immune response is a complex process that involves cell-cell interactions (Springer et al., Ann. Rev. Immunol., 5:223-252 (1987)), particularly between T and accessory cells such as APC's, and production of soluble immune mediators (cytokines or lymphokines) (Dinarello, New Engl. J. Med., 317:940-945 (1987); Sallusto, J. Exp. Med., 179:1109-1118 (1994)). This response is regulated by several T-cell surface receptors, including the T-cell receptor complex (Weiss, Ann. Rev. Immunol., 4:593-619 (1986)) and other “accessory” surface molecules (Allison, Curr. Opin. Immunol., 6:414-419 (1994); Springer (1987), supra). Many of these accessory molecules are naturally occurring cell surface differentiation (CD) antigens defined by the reactivity of monoclonal antibodies on the surface of cells (McMichael, ed., Leukocyte Typing Iff, Oxford Univ. Press, Oxford, N.Y. (1987)).

Early studies suggested that B lymphocyte activation requires two signals (Bretscher, Science, 169:1042-1049 (1970)) and now it is believed that all lymphocytes require two signals for their optimal activation, an antigen specific or clonal signal, as well as a second, antigen non-specific signal. (Janeway, supra). Freeman (J. Immunol., 143:2714-2722 (1989)) isolated and sequenced a cDNA clone encoding a B cell activation antigen recognized by MAb B7 (Freeman, J. Immunol., 138:3260 (1987)). COS cells transfected with this cDNA have been shown to stain by both labeled MAb B7 and MAb BB-1 (Clark, Human Immunol., 16:100-113 (1986); Yokochi, J. Immunol., 128:823 (1981); Freeman et al. (1989), supra; Freeman et al. (1987), supra). In addition, expression of this antigen has been detected on cells of other lineages, such as monocytes (Freeman et al. (1989), supra).

T helper cell (Th) antigenic response requires signals provided by APC's. The first signal is initiated by interaction of the T cell receptor complex (Weiss, J. Clin. Invest., 86:1015 (1990)) with antigen presented in the context of class II major histocompatibility complex (MHC) molecules on the APC (Allen, Immunol. Today, 8:270 (1987)). This antigen-specific signal is not sufficient to generate a full response, and in the absence of a second signal may actually lead to clonal inactivation or anergy (Schwartz, Science, 248:1349 (1990)). The requirement for a second “costimulatory” signal provided by the MHC has been demonstrated in a number of experimental systems (Schwartz, supra; Weaver et al., Immunol. Today, 11:49 (1990)).

CD28 antigen, a homodimeric glycoprotein of the immunoglobulin superfamily (Aruffo et al., Proc. Natl. Acad. Sci., 84:8573-8577 (1987)), is an accessory molecule found on most mature human T cells (Damle et al., J. Immunol., 131:2296-2300 (1983)). Current evidence suggests that this molecule functions in an alternative T cell activation pathway distinct from that initiated by the T-cell receptor complex (June et al., Mol. Cell. Biol., 7:4472-4481 (1987)). Monoclonal antibodies (MAbs) reactive with CD28 antigen can augment T cell responses initiated by various polyclonal stimuli (reviewed by June et al., supra). These stimulatory effects may result from MAb-induced cytokine production (Thompson et al., Proc. Natl. Acad. Sci., 86:1333-1337 (1989); and Lindsten et al., Science, 244:339-343 (1989)) as a consequence of increased mRNA stabilization (Lindsten et al. (1989), supra). Anti-CD28 mAbs can also have inhibitory effects, i.e., they can block autologous mixed lymphocyte reactions (Damle et al., Proc. Natl. Acad. Sci., 78:5096-6001 (1981)) and activation of antigen-specific T cell clones (Lesslauer et al., Eur. J. Immunol., 16:1289-1296 (1986)).

Some studies have indicated that CD28 is a counter-receptor for the B cell activation antigen, B7/BB-1 (Linsley et al., Proc. Natl. Acad. Sci. USA, 87:5031-5035 (1990)). The B7/BB-I antigen is hereafter referred to as the “B7 antigen”. The B7 ligands are also members of the immunoglobulin superfamily but have, in contrast to CD28, two Ig domains in their extracellular region, an N-terminal variable (V)-like domain followed by a constant (C)-like domain.

Delivery of a non-specific costimulatory signal to the T cell requires at least two homologous B7 family members found on APC's, B7-1 (also called B7, B7. 1, or CD80) and B7-2 (also called B7.2 or CD86), both of which can deliver costimulatory signals to T cells via CD28. Costimulation through CD28 promotes T cell activation.

CD28 has a single extracellular variable region (V)-like domain (Aruffo et al., supra). A homologous molecule, CTLA-4, has been identified by differential screening of a murine cytolytic-T cell cDNA library (Brunet, Nature, 328:267-270 (1987)).

CTLA-4 (CD152) is a T cell surface molecule that was originally identified by differential screening of a murine cytolytic T cell cDNA library (Brunet et al., Nature, 328:267-270 (1987)). CTLA-4 is also a member of the immunoglobulin (Ig) superfamily; CTLA-4 comprises a single extracellular Ig domain. Researchers have reported the cloning and mapping of a gene for the human counterpart of CTLA-4 (Dariavach et al., Eur. J. Immunol., 18:1901-1905 (1988)) to the same chromosomal region (2q33-34) as CD28 (Lafage-Pochitaloff et al., Immunogenetics, 31:198-201 (1990)). Sequence comparison between this human CTLA-4 DNA and that encoding CD28 proteins reveals significant homology of sequence, with the greatest degree of homology in the juxtamembrane and cytoplasmic regions (Brunet et al. (1988), supra; Dariavach et al. (1988), supra).

The CTLA-4 is inducibly expressed by T cells. It binds to the B7-family of molecules (primarily CD80 and CD86) on antigen-presenting cells (Chambers et al., Ann. Rev Immunol., 19:565-594 (2001)). When triggered, it inhibits T-cell proliferation and function. Mice genetically deficient in CTLA-4 develop lymphoproliferative disease and autoimmunity (Tivol et al., Immunity, 3:541-547 (1995)). In pre-clinical models, CTLA-4 blockade also augments anti-tumor immunity (Leach et al., Science, 271:1734-1736 (1996); van Elsas et al., J. Exp. Med., 190:355-366 (1999)). These findings led to the development of antibodies that block CTLA-4 for use in cancer immunotherapy.

Blockade of CTLA-4 by a monoclonal antibody leads to the expansion of all T cell populations, with activated CD4⁺ and CD8⁺ T cells mediating tumor cell destruction (Melero et al., Nat. Rev. Cancer, 7:95-106 (2007); Wolchok et al., The Oncologist, 13(Suppl. 4):2-9 (2008)). The antitumor response that results from the administration of anti-CTLA-4 antibodies is believed to be due to an increase in the ratio of effector T cells to regulatory T cells within the tumor microenvironment, rather than simply from changes in T cell populations in the peripheral blood (Quezada et al., J. Clin. Invest., 116:1935-1945 (2006)). One such agent under clinical investigation is Ipilimumab.

Ipilimumab (previously MDX-010; Medarex Inc.) is a fully human, anti-human CTLA-4 monoclonal antibody that blocks the binding of CTLA-4 to CD80 and CD86 expressed on antigen presenting cells, thereby, blocking the negative down-regulation of the immune responses elicited by the interaction of these molecules. Initial studies in patients with melanoma showed that Ipilimumab could cause objective durable tumor regressions (Phan et al., Proc. Natl. Acad. Sci. USA, 100:8372-8377 (2003)). Also, reductions of serum tumor markers were seen for some patients with ovarian or prostate cancer (Hodi et al., Proc. Natl. Acad. Sci. USA, 100:4712-4717 (2003)). More recently, Ipilimumab has demonstrated antitumor activity in patients with advanced melanoma (Weber et al., J. Clin. Oncol., 26:5950-5956 (2008); Weber, Cancer Immunol. Immunother., 58:823-830 (2009)).

Combination therapies for chemotherapeutic agents are increasing common for oncology indications. Often, such combination treatments are based upon pre-clinical data that demonstrate synergistic efficacy relative to either agent alone. As a result, most combination therapies are based upon concurrent, or close to concurrent, administration of one or more agents. While such synergistic treatment regimens represent an advance over the standard of care provided for each individually administered agent, deviation from concurrent treatment regimens is rare. As a result, there is a need in the art to identify optimal treatment regimens for any given combination. In particular, there is a need in the art to identify optimal treatment regimens for the combination of an immunotherapeutic agent with one or more chemotherapeutics.

The present inventors have discovered, for the first time, the sequential administration of one or more rounds of a chemotherapeutic agent followed by the administration of one or more rounds of an immunotherapeutic agent results in enhanced efficacy in the treatment of cancer.

### SUMMARY OF THE INVENTION

The present invention provides a method for treating a patient with cancer comprising the sequential administration of (i) one or more cycles of a chemotherapeutic agent, followed by (ii) one or more cycles of a combination comprising an immunomodulatory agent with said chemotherapeutic agent. In one aspect of the present invention, the immunomodulatory agent is a modulator of the co-stimulatory pathway. In another aspect of the present invention, the chemotherapeutic agent is a microtubulin stabilizing agent.

The present invention provides a method for treating a patient with cancer comprising the sequential administration of (i) one or more cycles of a chemotherapeutic agent, followed by (ii) one or more cycles of a combination comprising an immunomodulatory agent with said chemotherapeutic agent. In one aspect of the present invention, the immunomodulatory agent is a modulator of the co-stimulatory pathway, and is selected from the group consisting of: Ipilimumab; ORENCIA®; Belatacept; CD28 antagonists, CD80 antagonists, CD86 antagonists, PD1, PDL1, CD137, 41BB, and CTLA-4 antagonists. In another aspect of the present invention, the chemotherapeutic agent is one or more of the microtubulin stabilizing agents selected from the group consisting of: pacitaxel; carboplatin; an epothilone; ixabepilone; epothilone A; epothilone B; epothilone C; epothilone D; a taxane; Dacarbazine; PARAPLATIN®; and Docetaxel.

The present invention provides a method for treating a patient with cancer comprising the sequential administration of (i) one or more cycles of a chemotherapeutic agent, followed by (ii) one or more cycles of a combination comprising an immunomodulatory agent with said chemotherapeutic agent. In one aspect of the present invention, the immunomodulatory agent is a modulator of the co-stimulatory pathway, and is Ipilimumab. In another aspect of the present invention, the chemotherapeutic agent is the combination of pacitaxel and carboplatin.

The present invention provides a method for treating a patient with cancer comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes. In one aspect of the present invention, the immunomodulatory agent is a modulator of the co-stimulatory pathway. In another aspect of the present invention, the chemotherapeutic agent is a microtubulin stabilizing agent.

The present invention provides a method for treating a patient with cancer comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes. In one aspect of the present invention, the immunomodulatory agent is a modulator of the co-stimulatory pathway, and is selected from the group consisting of: Ipilimumab; ORENCIA®; Belatacept; CD28 antagonists, CD80 antagonists, CD86 antagonists, PD1, PDL1, CD137, 41BB, and CTLA-4 antagonists. In another aspect of the present invention, the chemotherapeutic agent is one or more of the microtubulin stabilizing agents selected from the group consisting of: pacitaxel; carboplatin; an epothilone; ixabepilone; epothilone A; epothilone B; epothilone C; epothilone D; a taxane; Dacarbazine; PARAPLATIN®; and Docetaxel.

The present invention provides a method for treating a patient with cancer comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes. In one aspect of the present invention, the immunomodulatory agent is a modulator of the co-stimulatory pathway, and is Ipilimumab. In another aspect of the present invention, the chemotherapeutic agent is pacitaxel or carboplatin; or the combination of pacitaxel and carboplatin.

The present invention provides a method for treating a patient with cancer comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: lung cancer; and non-small cell lung cancer. In one aspect of the present invention, the immunomodulatory agent is a modulator of the co-stimulatory pathway. In another aspect of the present invention, the chemotherapeutic agent is a microtubulin stabilizing agent.

The present invention provides a method for treating a patient with cancer comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: lung cancer; and non-small cell lung cancer. In one aspect of the present invention, the immunomodulatory agent is a modulator of the co-stimulatory pathway, and is selected from the group consisting of: Ipilimumab; ORENCIA®; Belatacept; CD28 antagonists, CD80 antagonists, CD86 antagonists, PD1, PDL1, CD137, 41BB, and CTLA-4 antagonists. In another aspect of the present invention, the chemotherapeutic agent is one or more of the microtubulin stabilizing agents selected from the group consisting of: pacitaxel; carboplatin; an epothilone; ixabepilone; epothilone A; epothilone B; epothilone C; epothilone D; a taxane; Dacarbazine; PARAPLATIN®; and Docetaxel.

The present invention provides a method for treating a patient with cancer comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: lung cancer; and non-small cell lung cancer. In one aspect of the present invention, the immunomodulatory agent is a modulator of the co-stimulatory pathway, and is Ipilimumab. In another aspect of the present invention, the chemotherapeutic agent is pacitaxel or carboplatin; or the combination of pacitaxel and carboplatin.

The present invention provides a method for treating a patient with cancer with a decreased likelihood of the patient having an adverse event, comprising the sequential administration of (i) one or more cycles of a chemotherapeutic agent, followed by (ii) one or more cycles of a combination comprising an immunomodulatory agent with said chemotherapeutic agent, wherein said sequential administration has a decreased likelihood of a patient having an adverse event relative to concurrent administration of said agent(s). In one aspect of the present invention, the immunomodulatory agent is a modulator of the co-stimulatory pathway. In another aspect of the present invention, the chemotherapeutic agent is a microtubulin stabilizing agent.

The present invention provides a method for treating a patient with cancer with a decreased likelihood of the patient having an adverse event, comprising the sequential administration of (i) one or more cycles of a chemotherapeutic agent, followed by (ii) one or more cycles of a combination comprising an immunomodulatory agent with said chemotherapeutic agent, wherein said sequential administration has a decreased likelihood of a patient having an adverse event relative to concurrent administration of said agent(s). In one aspect of the present invention, the immunomodulatory agent is a modulator of the co-stimulatory pathway, and is selected from the group consisting of: Ipilimumab; ORENCIA®; Belatacept; CD28 antagonists, CD80 antagonists, CD86 antagonists, PD1, PDL1, CD137, 41BB, and CTLA-4 antagonists. In another aspect of the present invention, the chemotherapeutic agent is one or more of the microtubulin stabilizing agents selected from the group consisting of: pacitaxel; carboplatin; an epothilone; ixabepilone; epothilone A; epothilone B; epothilone C; epothilone D; a taxane; Dacarbazine; PARAPLATIN®; and Docetaxel.

The present invention provides a method for treating a patient with cancer with a decreased likelihood of the patient having an adverse event, comprising the sequential administration of (i) one or more cycles of a chemotherapeutic agent, followed by (ii) one or more cycles of a combination comprising an immunomodulatory agent with said chemotherapeutic agent, wherein said sequential administration has a decreased likelihood of a patient having an adverse event relative to concurrent administration of said agent(s). In one aspect of the present invention, the immunomodulatory agent is a modulator of the co-stimulatory pathway, and is Ipilimumab. In another aspect of the present invention, the chemotherapeutic agent is pacitaxel or carboplatin; or the combination of pacitaxel and carboplatin.

The present invention provides a method for treating a patient with cancer with a decreased likelihood of the patient having an adverse event, comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes, wherein said sequential administration has a decreased likelihood of a patient having an adverse event relative to concurrent administration of said agent(s). In one aspect of the present invention, the immunomodulatory agent is a modulator of the co-stimulatory pathway. In another aspect of the present invention, the chemotherapeutic agent is a microtubulin stabilizing agent.

The present invention provides a method for treating a patient with cancer with a decreased likelihood of the patient having an adverse event, comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes, wherein said sequential administration has a decreased likelihood of a patient having an adverse event relative to concurrent administration of said agent(s). In one aspect of the present invention, the immunomodulatory agent is a modulator of the co-stimulatory pathway, and is selected from the group consisting of: Ipilimumab; ORENCIA®; Belatacept; CD28 antagonists, CD80 antagonists, CD86 antagonists, PD1, PDL1, CD137, 41BB, and CTLA-4 antagonists. In another aspect of the present invention, the chemotherapeutic agent is one or more of the microtubulin stabilizing agents selected from the group consisting of: pacitaxel; carboplatin; an epothilone; ixabepilone; epothilone A; epothilone B; epothilone C; epothilone D; a taxane; Dacarbazine;

PARAPLATIN®; and Docetaxel.

The present invention provides a method for treating a patient with cancer with a decreased likelihood of the patient having an adverse event, comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes, wherein said sequential administration has a decreased likelihood of a patient having an adverse event relative to concurrent administration of said agent(s). In one aspect of the present invention, the immunomodulatory agent is a modulator of the co-stimulatory pathway, and is Ipilimumab. In another aspect of the present invention, the chemotherapeutic agent is pacitaxel or carboplatin; or the combination of pacitaxel and carboplatin.

The present invention provides a method for treating a patient with cancer with a decreased likelihood of the patient having an adverse event, comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: lung cancer; and non-small cell lung cancer, wherein said sequential administration has a decreased likelihood of a patient having an adverse event relative to concurrent administration of said agent(s). In one aspect of the present invention, the immunomodulatory agent is a modulator of the co-stimulatory pathway. In another aspect of the present invention, the chemotherapeutic agent is a microtubulin stabilizing agent.

The present invention provides a method for treating a patient with cancer with a decreased likelihood of the patient having an adverse event, comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: lung cancer; and non-small cell lung cancer, wherein said sequential administration has a decreased likelihood of a patient having an adverse event relative to concurrent administration of said agent(s). In one aspect of the present invention, the immunomodulatory agent is a modulator of the co-stimulatory pathway, and is selected from the group consisting of: Ipilimumab; ORENCIA®; Belatacept; CD28 antagonists, CD80 antagonists, CD86 antagonists, PD1, PDL1, CD137, 41BB, and CTLA-4 antagonists. In another aspect of the present invention, the chemotherapeutic agent is one or more of the microtubulin stabilizing agents selected from the group consisting of: pacitaxel; carboplatin; an epothilone; ixabepilone; epothilone A; epothilone B; epothilone C; epothilone D; a taxane; Dacarbazine; PARAPLATIN®; and Docetaxel.

The present invention provides a method for treating a patient with cancer with a decreased likelihood of the patient having an adverse event, comprising the sequential administration of (i) one or more cycles of a chemotherapeutic agent, followed by (ii) one or more cycles of a combination comprising an immunomodulatory agent with said chemotherapeutic agent, wherein the cancer is selected from the group consisting of: lung cancer; and non-small cell lung cancer, wherein said sequential administration has a decreased likelihood of a patient having an adverse event relative to concurrent administration of said agent(s). In one aspect of the present invention, the immunomodulatory agent is a modulator of the co-stimulatory pathway, and is Ipilimumab. In another aspect of the present invention, the chemotherapeutic agent is pacitaxel or carboplatin; or the combination of pacitaxel and carboplatin.

The present invention provides a method for treating a patient with cancer with a sequential administration of (i) one or more cycles of a chemotherapeutic agent, followed by (ii) one or more cycles of a combination comprising an immunomodulatory agent with said chemotherapeutic agent, wherein said method optionally comprises an Intervening Period in-between (i) and (ii), wherein said Intervening Period is between 0 days to 24 weeks in time. In one aspect of the present invention, the Intervening Period is between 2 to 8 weeks. In one aspect of the present invention, the Intervening Period is between 3 to 6 weeks.

The present invention provides a method for treating a patient with cancer comprising the sequential administration of (i) one or more cycles of a chemotherapeutic agent, followed by (ii) one or more cycles of an immunomodulatory agent. In one aspect of the present invention, the immunomodulatory agent is a modulator of the co-stimulatory pathway. In another aspect of the present invention, the chemotherapeutic agent is a microtubulin stabilizing agent.

The present invention provides a method for treating a patient with cancer comprising the sequential administration of (i) one or more cycles of a chemotherapeutic agent, followed by (ii) one or more cycles of an immunomodulatory agent. In one aspect of the present invention, the immunomodulatory agent is a modulator of the co-stimulatory pathway, and is selected from the group consisting of: Ipilimumab; ORENCIA®; Belatacept; CD28 antagonists, CD80 antagonists, CD86 antagonists, PD1, PDL1, CD137, 41BB, and CTLA-4 antagonists. In another aspect of the present invention, the chemotherapeutic agent is one or more of the microtubulin stabilizing agents selected from the group consisting of: pacitaxel; carboplatin; an epothilone; ixabepilone; epothilone A; epothilone B; epothilone C; epothilone D; a taxane; Dacarbazine; PARAPLATIN®; and Docetaxel.

The present invention provides a method for treating a patient with cancer comprising the sequential administration of (i) one or more cycles of a chemotherapeutic agent, followed by (ii) one or more cycles of an immunomodulatory agent. In one aspect of the present invention, the immunomodulatory agent is a modulator of the co-stimulatory pathway, and is Ipilimumab. In another aspect of the present invention, the chemotherapeutic agent is pacitaxel or carboplatin; or the combination of pacitaxel and carboplatin.

The present invention provides a method for treating a patient with cancer comprising the sequential administration of (i) one or more cycles of a chemotherapeutic agent, followed by (ii) one or more cycles of an immunomodulatory agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes. In one aspect of the present invention, the immunomodulatory agent is a modulator of the co-stimulatory pathway. In another aspect of the present invention, the chemotherapeutic agent is a microtubulin stabilizing agent.

The present invention provides a method for treating a patient with cancer comprising the sequential administration of (i) one or more cycles of a chemotherapeutic agent, followed by (ii) one or more cycles of an immunomodulatory agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes. In one aspect of the present invention, the immunomodulatory agent is a modulator of the co-stimulatory pathway, and is selected from the group consisting of: Ipilimumab; ORENCIA®; Belatacept; CD28 antagonists, CD80 antagonists, CD86 antagonists, PD1, PDL1, CD137, 41BB, and CTLA-4 antagonists. In another aspect of the present invention, the chemotherapeutic agent is one or more of the microtubulin stabilizing agents selected from the group consisting of: pacitaxel; carboplatin; an epothilone; ixabepilone; epothilone A; epothilone B; epothilone C; epothilone D; a taxane; Dacarbazine; PARAPLATIN®; and Docetaxel.

The present invention provides a method for treating a patient with cancer comprising the sequential administration of (i) one or more cycles of a chemotherapeutic agent, followed by (ii) one or more cycles of an immunomodulatory agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes. In one aspect of the present invention, the immunomodulatory agent is a modulator of the co-stimulatory pathway, and is Ipilimumab. In another aspect of the present invention, the chemotherapeutic agent is the combination of pacitaxel and carboplatin.

The present invention provides a method for treating a patient with cancer comprising the sequential administration of (i) one or more cycles of a chemotherapeutic agent, followed by (ii) one or more cycles of an immunomodulatory agent, wherein the cancer is selected from the group consisting of: lung cancer; and non-small cell lung cancer. In one aspect of the present invention, the immunomodulatory agent is a modulator of the co-stimulatory pathway. In another aspect of the present invention, the chemotherapeutic agent is a microtubulin stabilizing agent.

The present invention provides a method for treating a patient with cancer comprising the sequential administration of (i) one or more cycles of a chemotherapeutic agent, followed by (ii) one or more cycles of an immunomodulatory agent, wherein the cancer is selected from the group consisting of: lung cancer; and non-small cell lung cancer. In one aspect of the present invention, the immunomodulatory agent is a modulator of the co-stimulatory pathway, and is selected from the group consisting of: Ipilimumab; ORENCIA®; Belatacept; CD28 antagonists, CD80 antagonists, CD86 antagonists, PD1, PDL1, CD137, 41BB, and CTLA-4 antagonists. In another aspect of the present invention, the chemotherapeutic agent is one or more of the microtubulin stabilizing agents selected from the group consisting of: pacitaxel; carboplatin; an epothilone; ixabepilone; epothilone A; epothilone B; epothilone C; epothilone D; a taxane; Dacarbazine; PARAPLATIN®; and Docetaxel.

The present invention provides a method for treating a patient with cancer comprising the sequential administration of (i) one or more cycles of a chemotherapeutic agent, followed by (ii) one or more cycles of an immunomodulatory agent, wherein the cancer is selected from the group consisting of: lung cancer; and non-small cell lung cancer. In one aspect of the present invention, the immunomodulatory agent is a modulator of the co-stimulatory pathway, and is Ipilimumab. In another aspect of the present invention, the chemotherapeutic agent is pacitaxel or carboplatin; or the combination of pacitaxel and carboplatin.

The present invention provides a method for treating a patient with cancer with a decreased likelihood of the patient having an adverse event, comprising the sequential administration of (i) one or more cycles of a chemotherapeutic agent, followed by (ii) one or more cycles of an immunomodulatory agent, wherein said sequential administration has a decreased likelihood of a patient having an adverse event relative to concurrent administration of said agent(s). In one aspect of the present invention, the immunomodulatory agent is a modulator of the co-stimulatory pathway. In another aspect of the present invention, the chemotherapeutic agent is a microtubulin stabilizing agent.

The present invention provides a method for treating a patient with cancer with a decreased likelihood of the patient having an adverse event, comprising the sequential administration of (i) one or more cycles of a chemotherapeutic agent, followed by (ii) one or more cycles of an immunomodulatory agent, wherein said sequential administration has a decreased likelihood of a patient having an adverse event relative to concurrent administration of said agent(s). In one aspect of the present invention, the immunomodulatory agent is a modulator of the co-stimulatory pathway, and is selected from the group consisting of: Ipilimumab; ORENCIA®; Belatacept; CD28 antagonists, CD80 antagonists, CD86 antagonists, PD1, PDL1, CD137, 41BB, and CTLA-4 antagonists. In another aspect of the present invention, the chemotherapeutic agent is one or more of the microtubulin stabilizing agents selected from the group consisting of: pacitaxel; carboplatin; an epothilone; ixabepilone; epothilone A; epothilone B; epothilone C; epothilone D; a taxane; Dacarbazine; PARAPLATIN®; and Docetaxel.

The present invention provides a method for treating a patient with cancer with a decreased likelihood of the patient having an adverse event, comprising the sequential administration of (i) one or more cycles of a chemotherapeutic agent, followed by (ii) one or more cycles of an immunomodulatory agent, wherein said sequential administration has a decreased likelihood of a patient having an adverse event relative to concurrent administration of said agent(s). In one aspect of the present invention, the immunomodulatory agent is a modulator of the co-stimulatory pathway, and is Ipilimumab. In another aspect of the present invention, the chemotherapeutic agent is pacitaxel or carboplatin; or the combination of pacitaxel and carboplatin.

The present invention provides a method for treating a patient with cancer with a decreased likelihood of the patient having an adverse event, comprising the sequential administration of (i) one or more cycles of a chemotherapeutic agent, followed by (ii) one or more cycles of an immunomodulatory agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes, wherein said sequential administration has a decreased likelihood of a patient having an adverse event relative to concurrent administration of said agent(s). In one aspect of the present invention, the immunomodulatory agent is a modulator of the co-stimulatory pathway. In another aspect of the present invention, the chemotherapeutic agent is a microtubulin stabilizing agent.

The present invention provides a method for treating a patient with cancer with a decreased likelihood of the patient having an adverse event, comprising the sequential administration of (i) one or more cycles of a chemotherapeutic agent, followed by (ii) one or more cycles of an immunomodulatory agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes, wherein said sequential administration has a decreased likelihood of a patient having an adverse event relative to concurrent administration of said agent(s). In one aspect of the present invention, the immunomodulatory agent is a modulator of the co-stimulatory pathway, and is selected from the group consisting of: Ipilimumab; ORENCIA®; Belatacept; CD28 antagonists, CD80 antagonists, CD86 antagonists, PD1, PDL1, CD137, 41BB, and CTLA-4 antagonists. In another aspect of the present invention, the chemotherapeutic agent is one or more of the microtubulin stabilizing agents selected from the group consisting of: pacitaxel; carboplatin; an epothilone; ixabepilone; epothilone A; epothilone B; epothilone C; epothilone D; a taxane; Dacarbazine; PARAPLATIN®; and Docetaxel.

The present invention provides a method for treating a patient with cancer with a decreased likelihood of the patient having an adverse event, comprising the sequential administration of (i) one or more cycles of a chemotherapeutic agent, followed by (ii) one or more cycles of an immunomodulatory agent, wherein the cancer is selected from the group consisting of: a solid tumor, lung cancer; non-small cell lung cancer; melanoma, metastatic melanoma, prostate cancer, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes, wherein said sequential administration has a decreased likelihood of a patient having an adverse event relative to concurrent administration of said agent(s). In one aspect of the present invention, the immunomodulatory agent is a modulator of the co-stimulatory pathway, and is Ipilimumab. In another aspect of the present invention, the chemotherapeutic agent is pacitaxel or carboplatin; or the combination of pacitaxel and carboplatin.

The present invention provides a method for treating a patient with cancer with a decreased likelihood of the patient having an adverse event, comprising the sequential administration of (i) one or more cycles of a chemotherapeutic agent, followed by (ii) one or more cycles of an immunomodulatory agent, wherein the cancer is selected from the group consisting of: lung cancer; and non-small cell lung cancer, wherein said sequential administration has a decreased likelihood of a patient having an adverse event relative to concurrent administration of said agent(s). In one aspect of the present invention, the immunomodulatory agent is a modulator of the co-stimulatory pathway. In another aspect of the present invention, the chemotherapeutic agent is a microtubulin stabilizing agent.

The present invention provides a method for treating a patient with cancer with a decreased likelihood of the patient having an adverse event, comprising the sequential administration of (i) one or more cycles of a chemotherapeutic agent, followed by (ii) one or more cycles of an immunomodulatory agent, wherein the cancer is selected from the group consisting of: lung cancer; and non-small cell lung cancer, wherein said sequential administration has a decreased likelihood of a patient having an adverse event relative to concurrent administration of said agent(s). In one aspect of the present invention, the immunomodulatory agent is a modulator of the co-stimulatory pathway, and is selected from the group consisting of: Ipilimumab; ORENCIA®; Belatacept; CD28 antagonists, CD80 antagonists, CD86 antagonists, PD1, PDL1, CD137, 41BB, and CTLA-4 antagonists. In another aspect of the present invention, the chemotherapeutic agent is one or more of the microtubulin stabilizing agents selected from the group consisting of: pacitaxel; carboplatin; an epothilone; ixabepilone; epothilone A; epothilone B; epothilone C; epothilone D; a taxane; Dacarbazine; PARAPLATIN®; and Docetaxel.

The present invention provides a method for treating a patient with cancer with a decreased likelihood of the patient having an adverse event, comprising the sequential administration of (i) one or more cycles of a chemotherapeutic agent, followed by (ii) one or more cycles of an immunomodulatory agent, wherein the cancer is selected from the group consisting of: lung cancer; and non-small cell lung cancer, wherein said sequential administration has a decreased likelihood of a patient having an adverse event relative to concurrent administration of said agent(s). In one aspect of the present invention, the immunomodulatory agent is a modulator of the co-stimulatory pathway, and is Ipilimumab. In another aspect of the present invention, the chemotherapeutic agent is pacitaxel or carboplatin; or the combination of pacitaxel and carboplatin.

The present invention provides a method for treating a patient with cancer with a sequential administration of (i) one or more cycles of a chemotherapeutic agent, followed by (ii) one or more cycles of an immunomodulatory agent, wherein said method optionally comprises an Intervening Period in-between (i) and (ii), wherein said Intervening Period is between 0 days to 24 weeks in time. In one aspect of the present invention, the Intervening Period is between 2 to 8 weeks. In one aspect of the present invention, the Intervening Period is between 3 to 6 weeks.

### BRIEF DESCRIPTION OF THE FIGURES/DRAWINGS

FIGS. 1A-B. Study Design and Randomization Outline. A) Provides an overview of the concurrent and sequential dosing regimens for investigating the combination of an immunomodulatory agent with a chemotherapeutic agent in the Phase II CA84041 clinical trial. B) Provides a visual schematic illustrating the sequential or “phased” dosing regimen of the CA84041 clinical trial based upon mouse xenograft models.

FIG. 2. Topline Final Primary Endpoint Summary. The primary objective of the CA84041 study was to compare immune-related progression free survival (irPFS) between subjects receiving the chemotherapeutic agents paclitaxel/carboplatin in combination with each of two schedules of the immunomodulatory agent Ipilimumab (concurrent or sequential schedule, respectively) and subjects receiving paclitaxel/carboplatin in combination with placebo in Stage IIb/IV NSCLC patients. As shown, both Ipilimumab regimens were superior to placebo under significance criteria (one-sided test with α=0.10). Improvement in irPFS was numerically greater in the sequential/phased arm but influence of imbalance in baseline patients characteristics cannot be ruled out. More early progression (within 6 weeks after randomization) occurred in the concomitant arm than in the placebo arm.

FIGS. 3A-B. Kaplan-Meier Plot of IRC-Determined Immune Related PFS irRC Criteria with Randomized NSCLC Subjects. As shown, preliminary results suggest improvement in irPFS was numerically greater in the sequential/phased arm.

FIG. 4. Topline Intermediate Secondary Endpoint Messages. A secondary objective of the CA84041 study was to compare progression free survival (PFS) between the concurrent (respectively sequential) and placebo regimens. As shown, only the sequential/phased regimen showed statistically significant efficacy v. placebo.

FIGS. 5A-B. Kaplan-Meier Plot of IRC-Determined PFS per mWHO Criteria with Randomized NSCLC Subjects. As shown, only the sequential/phased regimen showed statistically significant efficacy v. placebo. The dashed arrow shows that Ipilimumab vs. placebo was initiated at 6 weeks for the phased schedule. For the first 6 weeks of the phased schedule, both treatment arms received paclitaxel/carboplatin only.

FIGS. 6A-B. Kaplan-Meier Plot of Duration of Immune-Related

Response per irRC Criteria with Randomized NSCLC Subjects with IRC-Determined irBOR of irCR or irPR per irRC Criteria. As shown, preliminary data suggested the concurrent regimen provided a longer duration of immune-related response than that observed for the sequential/phased regimen. The dashed arrow shows that Ipilimumab vs. placebo was initiated at 6 weeks for the phased schedule. For the first 6 weeks of the phased schedule, both treatment arms received paclitaxel/carboplatin only.

FIG. 7. Intermediate Differential Discontinuation of Ipilimumab/Placebo—Treated NSCLC Subjects. As shown, subjects in the concurrent arm differentially discontinued Ipilimumab/placebo (separately from other study drugs) at a numerically higher rate than sequential or placebo arms.

FIG. 8. Intermediate Discontinuation of All Study Therapy—Treated NSCLC Subjects. As shown, overall survival trends in favor of the sequential/phased reasons for final discontinuation of all study drugs were similar across treatment arms, with more concurrent arm patients withdrawing due to adverse events.

FIGS. 9A-B. Kaplan-Meier Plot of Overall Survival based upon an interim analysis of data from the CA1840141 study. As shown, overall survival trends in favor of the sequential/phased arm

FIGS. 10A-B. Kaplan-Meier Plot of Overall Survival based upon a final analysis of data from the CA1840141 study. As shown, overall survival trends in favor of the sequential/phased arm.

FIG. 11. Final Response Rate and Disease Control Rate based upon a final analysis of data from the CA1840141 study. The sequential/phased arm showed a higher rate of Immune-Related Best Overall Response Rate (irBORR), the highest Best Overall Response Rate using mWHO criteria, the highest Immune-Related Disease Control Rate (irDCR), and the highest Disease Control Rate using mWHO criteria.

FIG. 12. Final Discontinuation of All Study Therapy—Treated NSCLC Subjects. As shown, overall survival trends in favor of the sequential/phased reasons for final discontinuation of all study drugs were similar across treatment arms, with more concurrent arm patients withdrawing due to adverse events.

FIG. 13. Final Analysis of Common Drug-Related Adverse Events. As shown, the phased/sequential arm had a lower level of incidence of grade 3 and grade 4 adverse events relative to the concurrent arm.

FIG. 14. Final Analysis of Key Immune-Related Adverse Events. As shown, the phased/sequential arm had a lower level of incidence of grade 3 immune related adverse events, with an elevated rate of grade 4 adverse events relative to the concurrent arm.

### DETAILED DESCRIPTION OF THE INVENTION

The present invention is based, in part, on data from a phase II clinical trial that expectedly demonstrated patients who were sequentially administered one or more cycles of a chemotherapeutic agent followed by one or more cycles of a combination comprising an immunomodulatory agent with a chemotherapeutic agent exhibited superior responses relative to concurrently administering these agents. Specifically, patients within the sequential arm of the study showed better immune-related progressive free survival; statistically significant progression free survival; improved immune-related best overall response rate; lower rates of adverse events, higher tolerances to chemotherapeutic agent exposure; and lower rates of study discontinuation, relative to patients in the concurrent arm of the study.

The teachings of the present invention are believed to be the first association between the sequential administration of a chemotherapeutic agent followed by a combination comprising a chemotherapeutic agent and an immunotherapeutic agent with increased outcomes in terms of efficacy, safety, and tolerability.

The combination of a chemotherapeutic agent with an immunotherapeutic agent has been previously described. However, the standard dosing regimens have been devoted to administering a chemotherapeutic agent with an immunotherapeutic agent concurrently, but have not previously described the sequential administration of a chemotherapeutic agent followed by of a combination comprising an immunomodulatory agent with a chemotherapeutic agent. In addition, the sequential administration of a chemotherapeutic agent followed by an immunotherapeutic agent has similarly not been described. The present invention supports both of these novel dosing regimens.

For the purposes of the present invention, the sequential administration of one or more cycles of a chemotherapeutic agent followed by one or more cycles of either the combination comprising a chemotherapeutic agent and an immunomodulatory agent, or simply an immunomodulatory agent, may optionally comprise an “Intervening Period”, defined as a time period beginning from the end of the last chemotherapeutic cycle up until the beginning of the first immunomodulatory cycle, either concurrently with the last cycle of the chemotherapeutic agent, or sequentially at the end of the one or more chemotherapeutic agent cycle(s). The intervening Period may be about 24 weeks. In another embodiment of the present invention, the intervening Period may be about 20 weeks. In another embodiment of the present invention, the intervening Period may be about 18 weeks. In another embodiment of the present invention, the intervening Period may be about 15 weeks. In another embodiment of the present invention, the intervening Period may be about 12 weeks. In another embodiment of the present invention, the intervening Period may be about 11 weeks. In another embodiment of the present invention, the intervening Period may be about 10 weeks. In another embodiment of the present invention, the intervening Period may be about 9 weeks. In another embodiment of the present invention, the intervening Period may be about 8 weeks. In another embodiment of the present invention, the intervening Period may be about 7 weeks. In another embodiment of the present invention, the intervening Period may be about 6 weeks. In another embodiment of the present invention, the intervening Period may be about 5 weeks. In another embodiment of the present invention, the intervening Period may be about 4 weeks. In another embodiment of the present invention, the intervening Period may be about 3 weeks. In another embodiment of the present invention, the intervening Period may be about 2 weeks. In another embodiment of the present invention, the intervening Period may be about 1 week. In another embodiment of the present invention, the intervening Period may be about 1, 2, 3, 4, 5, 6, or 7 days. In this context, the term “about” shall be construed to mean±1, 2, 3, 4, 5, 6, or 7 days more or less than the stated intervening Period.

In one embodiment of the present invention, the Intervening Period is between 2 to 8 weeks. In another embodiment of the present invention, the

Intervening Period is between 3 to 6 weeks.

In another embodiment of the present invention, the Intervening Period may be less than 0 days such that the immunomodulatory agent is administered concurrently with the last cycle of the chemotherapeutic agent.

In another embodiment of the present invention, the Intervening Period may be 0 days such that either the immunomodulatory agent, or a combination comprising an immunomodulatory agent and one or more chemotherapeutic agents, is administered immediately following the last day of the last cycle of the chemotherapeutic agent.

The phrase “immunomodulatory cycle” or “cycle of an immunomodulatory agent” is meant to encompass either one or more dosing cycle(s) of an immunomodulatory agent, or one or more dosing cycle(s) of a combination comprising an immunomodulatory agent and one or more chemotherapeutic agents.

For the purposes of the present invention, “one or more cycles of a chemotherapeutic agent” and/or “one or more cycles of an immunomodulatory agent” means at least 1, at least 2, at least 3, at least 4, at least 5, at least 6, at least 7, at least 8, at least 9, or at least 10 cycles of primary treatment with either agent(s), followed by one or more optional maintenance cycles of either agent(s). The maintenance cycle(s) may follow a similar number of cycles as outlined for the primary therapy, or may be significantly longer or shorter in terms of cycle number, depending upon the patient's disease and/or severity.

In preferred embodiments of the present invention, the phrase “one or more cycles of a chemotherapeutic agent” is meant to encompass one or more cycles of either a chemotherapeutic agent or a combination of one or more chemotherapeutic agents. In one embodiment, “one or more cycles of a chemotherapeutic agent” means more than two cycles.

In another aspect of the present invention, the sequential dosing regimen may comprise a “hybrid cycle” in which the patient is administered one or more chemotherapeutic agent cycles, followed by one or more immunomodulatory cycles, followed by one or more chemotherapeutic agent cycles and/or one or more immunomodulatory cycles.

The phrase “sequential dosing regimen”, generally refers to treating a patient with at least two cycles of an agent in a specific order, wherein one cycle is administered after the other. In addition, the phrase “sequential dosing regimen” also encompasses the phrase “phased dosing regimen” as it is traditionally referred to in the pharmaceutical arts. In one context, “sequential dosing regimen” refers to not only the order in which the cycles are administered, but also to the entire treatment regimen for the patient. For example, “sequential dosing regimen” may include the complete dosing regimen for the patient including one or more cycles of a chemotherapeutic agent, followed by one or more cycles of either an immunomodulatory agent or a combination comprising an immunomodulatory agent and one or more chemotherapeutic agents.

For the purposes of the present invention, the sequential administration of a chemotherapeutic agent followed by an immunomodulatory agent, or a combination comprising an immunomodulatory agent and one or more chemotherapeutic agents, is not meant to include the immediate administration of an immunomodulatory agent after failure of an initial chemotherapeutic agent treatment as the cancer patient's primary therapy. Rather, the sequential dosing regimen of the present invention is intended as a stand-alone, primary therapy that includes the sequential administration of a chemotherapeutic agent followed by an immunomodulatory agent, or a combination comprising an immunomodulatory agent and one or more chemotherapeutic agents (i.e., either of which referred to as an “immunomodulatory cycle”). However, the sequential dosing regimen of the present invention may be administered after a sufficient period of time after prior chemotherapeutic therapy has passed, which may be at least about 3 weeks, about 4 weeks, about 5 weeks, about 6 weeks, about 7 weeks, about 8 weeks, about 9 weeks, about 10 weeks, about 11 weeks, about 12 weeks, or more weeks after prior chemotherapeutic therapy has ended and/or after the physician has determined the prior chemotherapeutic therapy had failed.

In one embodiment of the present invention, the sequential dosing regimen comprises one chemotherapeutic cycle followed by one or more cycles of an immunotherapeutic agent, or a combination comprising an immunomodulatory agent and one or more chemotherapeutic agents.

In one embodiment of the present invention, the sequential dosing regimen comprises more than one chemotherapeutic cycle followed by one or more cycles of an immunotherapeutic agent, or a combination comprising an immunomodulatory agent and one or more chemotherapeutic agents.

In one embodiment of the present invention, the sequential dosing regimen comprises more than two chemotherapeutic cycles followed by one or more cycles of an immunotherapeutic agent, or a combination comprising an immunomodulatory agent and one or more chemotherapeutic agents.

In one embodiment of the present invention, the sequential dosing regimen comprises more than three chemotherapeutic cycles followed by one or more cycles of an immunotherapeutic agent, or a combination comprising an immunomodulatory agent and one or more chemotherapeutic agents.

In one embodiment of the present invention, the sequential dosing regimen comprises more than four chemotherapeutic cycles followed by one or more cycles of an immunotherapeutic agent, or a combination comprising an immunomodulatory agent and one or more chemotherapeutic agents.

In one embodiment of the present invention, the sequential dosing regimen comprises more than five chemotherapeutic cycles followed by one or more cycles of an immunotherapeutic agent, or a combination comprising an immunomodulatory agent and one or more chemotherapeutic agents.

In one embodiment of the present invention, the sequential dosing regimen comprises more than six chemotherapeutic cycles followed by one or more cycles of an immunotherapeutic agent, or a combination comprising an immunomodulatory agent and one or more chemotherapeutic agents.

The phrase “clinical benefit” or “benefit” refers to a condition where a patient achieves a complete response; partial response; stable disease; or as otherwise described herein.

The phrase “immunomodulatory agent” generally refers to an agent that either increases or decreases the function of the immune system, and/or as defined elsewhere herein, and includes co-stimulatory pathway modulators, Ipilimumab; ORENCIA®; Belatacept; CD28 antagonists, CD80 antagonists, CD86 antagonists, PD1, PDL1, CD137, 41BB, and CTLA-4 antagonists, among others disclosed herein.

The phrase “co-stimulatory pathway modulator”, generally refers to an immunomodulatory agent that functions by increasing or decreasing the function of the immune system by modulating the co-stimulatory pathway. In one aspect of the present invention, a co-stimulatory pathway modulator is an immunostimulant or T-cell activator, and may also encompass any agent that is capable of disrupting the ability of CD28 antigen to bind to its cognate ligand, to inhibit the ability of CTLA-4 to bind to its cognate ligand, to augment T cell responses via the co-stimulatory pathway, to disrupt the ability of B7 to bind to CD28 and/or CTLA-4, to disrupt the ability of B7 to activate the co-stimulatory pathway, to disrupt the ability of CD80 to bind to CD28 and/or CTLA-4, to disrupt the ability of CD80 to activate the co-stimulatory pathway, to disrupt the ability of CD86 to bind to CD28 and/or CTLA-4, to disrupt the ability of CD86 to activate the co-stimulatory pathway, and to disrupt the co-stimulatory pathway, in general from being activated. This necessarily includes small molecule inhibitors of CD28, CD80, CD86, CTLA-4, among other members of the co-stimulatory pathway; antibodies directed to CD28, CD80, CD86, CTLA-4, among other members of the co-stimulatory pathway; antisense molecules directed against CD28, CD80, CD86, CTLA-4, among other members of the co-stimulatory pathway; adnectins directed against CD28, CD80, CD86, CTLA-4, among other members of the co-stimulatory pathway, RNAi inhibitors (both single and double stranded) of CD28, CD80, CD86, CTLA-4, among other members of the co-stimulatory pathway, among other anti-CTLA-4 antagonists.

Suitable anti-CTLA-4 antagonist agents for use in the methods of the invention, include, without limitation, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, modulators of the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B1. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication No. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145):Abstract No. 2505 (2004) (antibody CP-675206); Mokyr et al., Cancer Res., 58:5301-5304 (1998), U.S. Pat. Nos. 5,977,318, 6,682,736, 7,109,003, and 7,132,281. Each of these references is specifically incorporated herein by reference for purposes of description of CTLA-4 antibodies. A preferred clinical CTLA-4 antibody is human monoclonal antibody 10D1 (also referred to as MDX-010 and Ipilimumab and available from Medarex, Inc., Bloomsbury, N.J.), disclosed in WO 01/14424.

As is known in the art, Ipilimumab refers to an anti-CTLA-4 antibody, and is a fully human IgG₁ antibody derived from transgenic mice having human genes encoding heavy and light chains to generate a functional human repertoire. Ipilimumab can also be referred to by its CAS Registry No. 477202-00-9, and is disclosed as antibody 10DI in PCT Publication No. WO 01/14424, incorporated herein by reference in its entirety and for all purposes. Specifically, Ipilimumab describes a human monoclonal antibody or antigen-binding portion thereof that specifically binds to CTLA-4, comprising a light chain variable region and a heavy chain variable region having a light chain variable region comprised of SEQ ID NO:1, and comprising a heavy chain region comprised of SEQ ID NO:2. Pharmaceutical compositions of Ipilimumab include all pharmaceutically acceptable compositions comprising Ipilimumab and one or more diluents, vehicles and/or excipients. Examples of a pharmaceutical composition comprising Ipilimumab are provided in PCT Publication No. WO2007/67959. Ipilimumab may be administered by I.V.

Light chain variable region for Ipilimumab:
(SEQ ID NO: 1)
EIVLTQSPGTLSLSPGERATLSCRASQSVGSSYLAWYQQKPGQAPRLLIY
GAFSRATGIPDRFSGSGSGTDFTLTISRLEPEDFAVYYCQQYGSSPWTFG
QGTKVEIK
Heavy chain variable region for Ipilimumab:
(SEQ ID NO: 2)
QVQLVESGGGVVQPGRSLRLSCAASGFTFSSYTMHWVRQAPGKGLEWVTF
ISYDGNNKYYADSVKGRFTISRDNSKNTLYLQMNSLRAEDTAIYYCARTG
WLGPFDYWGQGTLVTVSS

As noted elsewhere herein, the sequential administration of a chemotherapeutic agent followed by an immunomodulatory agent, or a combination comprising an immunomodulatory agent and one or more chemotherapeutic agents, may be administered either alone or in combination with a peptide antigen (e.g., gp100). A non-limiting example of a peptide antigen would be a gp100 peptide comprising, or alternatively consisting of, the sequence selected from the group consisting of: IMDQVPFSV (SEQ ID NO:3), and YLEPGPVTV (SEQ ID NO:4). Such a peptide may be administered orally, or preferably at 1 mg emulsified in incomplete Freund's adjuvant (IFA) injected s.c. in one extremity, and 1 mg of either the same or a different peptide emulsified in IFA may be injected in another extremity.

Disorders for which the sequential dosing regimens of the present invention may be useful in treating include, but are not limited to: melanoma, primary melanoma, unresectable stage III or IV malignant melanoma, lung cancer, non-small cell lung cancer, small cell lung cancer, prostate cancer; solid tumors, pancreatic cancer, prostatic neoplasms, breast cancer, neuroblastoma, kidney cancer, ovarian cancer, sarcoma, bone cancer, testicular cancer, hematopoietic cancers, leukemia, lymphoma, multiple myeloma, and myelodysplastic syndromes.

Additional disorders for which the sequential dosing regimens of the present invention may be useful in treating include, but are not limited to the following: glioma, gastrointestinal cancer, renal cancer, ovarian cancer, liver cancer, colorectal cancer, endometrial cancer, kidney cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, Philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include uticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof.

The terms “treating”, “treatment” and “therapy” as used herein refer to curative therapy, prophylactic therapy, preventative therapy, and mitigating disease therapy.

The phrase “more aggressive dosing regimen” or “increased dosing frequency regimen”, as used herein refers to a dosing regimen that necessarily exceeds the basal and/or prescribed dosing regimen of either the co-stimulatory pathway modulator, preferably Ipilimumab, arm of the sequential dosing regimen and/or the chemotherapeutic agent arm of the sequential dosing regimen, either due to an increased dosing frequency (about once a week, about bi-weekly, about once daily, about twice daily, etc.), increased or escalated dose (about 11, about 12, about 13, about 14, about 15, about 16, about 17, about 18, about 19, about 20, about 21, about 22, about 23, about 24, about 25, about 26, about 27, about 28, about 29, about 30, about 35, about 40 mg/ml), or by changing the route of administration which may result in an increased, bio-available level of said co-stimulatory modulator and/or said chemotherapeutic agent.

It is to be understood this invention is not limited to particular methods, reagents, compounds, compositions, or biological systems, which can, of course, vary. It is also to be understood that the terminology used herein is for the purpose of describing particular aspects only, and is not intended to be limiting.

As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, preferably ±5%, or ±1%, or as little as ±0.1% from the specified value, as such variations are appropriate to perform the disclosed methods, unless otherwise specified herein.

Specific sequential dosing regimens for any given patient may be established based upon the specific disease for which the patient has been diagnosed, or in conjunction with the stage of the patients disease. For example, if a patient is diagnosed with a less-aggressive cancer, or a cancer that is in its early stages, the patient may have an increased likelihood of achieving a clinical benefit and/or immune-related response to a typical sequential administration of a chemotherapeutic agent followed by an immunomodulatory agent. Alternatively, if a patient is diagnosed with a more-aggressive cancer, or a cancer that is in its later stages, the patient may have a decreased likelihood of achieving a clinical benefit and/or immune-related response to a typical sequential administration of a chemotherapeutic agent followed by an immunomodulatory agent, or a combination comprising an immunomodulatory agent and one or more chemotherapeutic agents, and thus may suggest that either higher doses of the immunomodulatory agent and/or chemotherapeutic agent therapy should be administered or more aggressive dosing regimens or either agent or combination therapy may be warranted. In one aspect, an increased dosing level of a immunomodulatory agent, such as Ipilimumab, would be about 10, 20, 30, 40, 50, 60, 70, 80, 90, or 95% more than the typical immunomodulatory agent dose for a particular indication or individual (e.g., about 0.3 mg/kg, about 3 mg/kg, about 10 mg/kg, about 15 mg/kg, about 20 mg/kg, about 25mg/kg, about 30 mg/kg), or about 1.5×, 2×, 2.5×, 3×, 3.5×, 4×, 4.5×, 5×, 6×, 7×, 8×, 9×, or 10× more immunomodulatory agent than the typical co-stimulatory pathway modulator dose for a particular indication or for individual. In another aspect, an increased dosing level of a chemotherapeutic agent would be about 10, 20, 30, 40, 50, 60, 70, 80, 90, or 95% more than the typical chemotherapeutic agent dose for a particular indication or individual (e.g., about 0.3 mg/kg, about 3 mg/kg, about 10 mg/kg, about 15 mg/kg, about 20 mg/kg, about 25 mg/kg, about 30 mg/kg), or about 1.5×, 2×, 2.5×, 3×, 3.5×, 4×, 4.5×, 5×, 6×, 7×, 8×, 9×, or 10× more chemotherapeutic agent than the typical dose for a particular indication or for individual.

A therapeutically effective amount of co-stimulatory pathway modulator, preferably Ipilimumab, can be orally administered if it is a small molecule modulator, for example, or preferably injected into the patient, for example if it is a biologic agent. The actual dosage employed can be varied depending upon the requirements of the patient and the severity of the condition being treated. Determination of the proper starting dosage for a particular situation is within the skill of the art, though the assignment of a treatment regimen will benefit from taking into consideration the indication and the stage of the disease. Nonetheless, it will be understood that the specific dose level and frequency of dosing for any particular patient can be varied and will depend upon a variety of factors including the activity of the specific compound employed, the metabolic stability and length of action of that compound, the species, age, body weight, general health, sex and diet of the patient, the mode and time of administration, rate of excretion, drug combination, and severity of the particular condition. Preferred patients for treatment include animals, most preferably mammalian species such as humans, and domestic animals such as dogs, cats, and the like, patient to cancer.

The terms “combination” and “combinations” as used herein refer to either the chemotherapeutic agent or immunomodulatory agent; or to a more complex, sequential combination, which may include for example, the combination of either the immunotherapeutic agent or the chemotherapeutic agent with another immunotherapeutic agent or co-stimulatory pathway modulator, preferably an agonist (i.e., immunostimulant), PROVENGE®, a tubulin stabilizing agent (e.g., pacitaxol, epothilone, taxane, etc.), Bevacizumab, IXEMPRA®, Dacarbazine, PARAPLATIN®, Docetaxel, one or more peptide vaccines, MDX-1379 Melanoma Peptide Vaccine, one or more gp100 peptide vaccine, fowlpox-PSA-TRICOMT™ vaccine, vaccinia-PSA-TRICOMT™ vaccine, MART-1 antigen, sargramostim, ticilimumab, Combination Androgen Ablative Therapy; the combination of Ipilimumab and another co-stimulatory pathway modulator; combination of Ipilimumab and a tubulin stabilizing agent (e.g., pacitaxol, epothilone, taxane, etc.); combination of Ipilimumab and IXEMPRA® the combination of Ipilimumab with Dacarbazine, the combination of Ipilimumab with PARAPLATIN®, the combination of Ipilimumab with Docetaxel, the combination of Ipilimumab with one or more peptide vaccines, the combination of Ipilimumab with MDX-1379 Melanoma Peptide Vaccine, the combination of Ipilimumab with one or more gp100 peptide vaccine, the combination of Ipilimumab with fowlpox-PSA-TRICOM™ vaccine, the combination of Ipilimumab with vaccinia-PSA-TRICOMT™ vaccine, the combination of Ipilimumab with MART-1 antigen, the combination of Ipilimumab with sargramostim, the combination of Ipilimumab with ticilimumab, and/or the combination of Ipilimumab with Combination Androgen Ablative Therapy. The combinations of the present invention may also be used in conjunction with other well known therapies that are selected for their particular usefulness against the condition that is being treated. Such combinations may provide therapeutic options to those patients who present with more aggressive indications.

In another embodiment of the present invention, combination between an immunomodulatory agent and at least one other agent may comprise one or more of the following combinations, preferably administered sequentially in any order: Ipilimumab and TAXOL® and PARAPLATIN® (concurrent administration); Ipilimumab and TAXOL® and PARAPLATIN® (sequential administration); Ipilimumab and Dacarbazine; Ipilimumab and Bevacizumab; Ipilimumab and Budesonide; Ipilimumab and an inhibitor of CD137; and Ipilimumab and steroids (corticosteroids and the like).

In another embodiment of the present invention, the combination between an immunomodulatory agent and at least one other agent may comprise the following: agatolimod, belatacept, blinatumomab, CD40 ligand, anti-B7-1 antibody, anti-B7-2 antibody, anti-B7-H4 antibody, AG4263, eritoran, anti-CD137 monoclonal antibodies, anti-OX40 antibody, ISF-154, and SGN-70.

A variety of chemotherapeutics are known in the art, some of which are described herein. One type of chemotherapeutic is referred to as a metal coordination complex. It is believed this type of chemotherapeutic forms predominantly inter-strand DNA cross links in the nuclei of cells, thereby preventing cellular replication. As a result, tumor growth is initially repressed, and then reversed. Another type of chemotherapeutic is referred to as an alkylating agent. These compounds function by inserting foreign compositions or molecules into the DNA of dividing cancer cells. As a result of these foreign moieties, the normal functions of cancer cells are disrupted and proliferation is prevented. Another type of chemotherapeutic is an antineoplastic agent. This type of agent prevents, kills, or blocks the growth and spread of cancer cells. Still other types of anticancer agents include nonsteroidal aromastase inhibitors, bifunctional alkylating agents, etc.

In another embodiment of the present invention, the chemotherapeutic agent may comprise microtubule-stabilizing agents, such as ixabepilone (IXEMPRA®) and paclitaxel (TAXOL®), which commonly are used for the treatment of many types of cancer and represent an attractive class of agents to combine with CTLA-4 blockade.

The phrase “microtubulin modulating agent” is meant to refer to agents that either stabilize microtubulin or destabilize microtubulin synthesis and/or polymerization.

One microtubulin modulating agent is paclitaxel (marketed as TAXOL®), which is known to cause mitotic abnormalities and arrest, and promotes microtubule assembly into calcium-stable aggregated structures resulting in inhibition of cell replication.

Epothilones mimic the biological effects of TAXOL®, (Bollag et al., Cancer Res., 55:2325-2333 (1995), and in competition studies act as competitive inhibitors of TAXOL® binding to microtubules. However, epothilones enjoy a significant advantage over TAXOL® in that epothilones exhibit a much lower drop in potency compared to TAXOL® against a multiple drug-resistant cell line (Bollag et al. (1995)). Furthermore, epothilones are considerably less efficiently exported from the cells by P-glycoprotein than is TAXOL® (Gerth et al. (1996)). Additional examples of epothilones are provided in co-owned, PCT Application No. PCT/US2009/030291, filed Jan. 7, 2009, which is hereby incorporated by reference herein in its entirety for all purposes.

Ixabepilone is a semi-synthetic lactam analogue of patupilone that binds to tubulin and promotes tubulin polymerisation and microtubule stabilization, thereby arresting cells in the G2/M phase of the cell cycle and inducing tumor cell apoptosis.

Additional examples of microtubule modulating agents useful in combination with immunotherapy include, but are not limited to, allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (TAXOL®, NSC 125973), TAXOL® derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, epothilone C, epothilone D, desoxyepothilone A, desoxyepothilone B, [1S-[1R*,3R*(E),7R*,10S*,11R*,12R*,16S*]]-7-11-dihydroxy-8,8,10,12,16-pentamethyl-3-[1-methyl-2-(2-methyl-4-thiazolyl)ethenyl]-4-aza-17 oxabicyclo [14.1.0]heptadecane-5,9-dione (disclosed in U.S. Pat. No. 6,262,094, issued Jul. 17, 2001), [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione (disclosed in U.S. Ser. No. 09/506,481 filed on Feb. 17, 2000, and Examples 7 and 8 herein), and derivatives thereof; and other microtubule-disruptor agents. Additional antineoplastic agents include, discodermolide (see Service, Science, 274:2009 (1996)) estramustine, nocodazole, MAP4, and the like. Examples of such agents are also described in the scientific and patent literature, see, e.g., Bulinski, J. Cell Sci., 110:3055-3064 (1997); Panda, Proc. Natl. Acad. Sci. USA, 94:10560-10564 (1997); Muhlradt, Cancer Res., 57:3344-3346 (1997); Nicolaou, Nature, 387:268-272 (1997); Vasquez, Mol. Biol. Cell., 8:973-985 (1997); Panda, J. Biol. Chem., 271:29807-29812 (1996).

The following sets forth preferred therapeutic combinations and exemplary dosages for use in the methods of the present invention.

| | Dosage | |
|---|---|---|
| Sequential Therapeutic Combination(s) | mg/m² (per dose) | |
| Ixabepilone + | 1-500 | mg/m2 |
| anti-CTLA-4 Antibody | 0.1-25 | mg/kg |
| Paclitaxel + | 40-250 | mg/m2 |
| anti-CTLA-4 Antibody | 0.1-25 | mg/kg |
| Paclitaxel \| Carboplatin + | 40-250 mg/m2 \| 2-8 AUC | |
| anti-CTLA-4 Antibody | 0.1-25 | mg/kg |

While this table provides exemplary dosage ranges of co-stimulatory pathway modulators and certain anticancer agents of the invention, when formulating the pharmaceutical compositions of the invention the clinician may utilize preferred dosages as warranted by the condition of the patient being treated. For example, ixabepilone may preferably be administered at about 40 mg/m2 every 3 weeks. Paclitaxel may preferably be administered at about 135-175 mg/m2 every three weeks.

The anti-CTLA-4 antibody may preferably be administered at about 0.3-10 mg/kg, or the maximum tolerated dose. In an embodiment of the invention, a dosage of CTLA-4 antibody is administered about every three weeks. Alternatively, the CTLA-4 antibody may be administered by an escalating dosage regimen including administering a first dosage of CTLA-4 antibody at about 3 mg/kg, a second dosage of CTLA-4 antibody at about 5 mg/kg, and a third dosage of CTLA-4 antibody at about 9 mg/kg.

In another specific embodiment, the escalating dosage regimen includes administering a first dosage of CTLA-4 antibody at about 5 mg/kg and a second dosage of CTLA-4 antibody at about 9 mg/kg.

Further, the present invention provides an escalating dosage regimen, which includes administering an increasing dosage of CTLA-4 antibody about every six weeks.

In an aspect of the present invention, a stepwise escalating dosage regimen is provided, which includes administering a first CTLA-4 antibody dosage of about 3 mg/kg, a second CTLA-4 antibody dosage of about 3 mg/kg, a third CTLA-4 antibody dosage of about 5 mg/kg, a fourth CTLA-4 antibody dosage of about 5 mg/kg, and a fifth CTLA-4 antibody dosage of about 9 mg/kg. In another aspect of the present invention, a stepwise escalating dosage regimen is provided, which includes administering a first dosage of 5 mg/kg, a second dosage of 5 mg/kg, and a third dosage of 9 mg/kg.

The actual dosage employed may be varied depending upon the requirements of the patient and the severity of the condition being treated. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In practicing the many aspects of the invention herein, biological samples can be selected preferably from blood, blood cells (red blood cells or white blood cells). Cells from a sample can be used, or a lysate of a cell sample can be used. In certain embodiments, the biological sample comprises blood cells.

Pharmaceutical compositions for use in the present invention can include compositions comprising one or a combination of co-stimulatory pathway modulators in an effective amount to achieve the intended purpose. A therapeutically effective dose refers to that amount of active ingredient which ameliorates the symptoms or condition. Therapeutic efficacy and toxicity in humans can be predicted by standard pharmaceutical procedures in cell cultures or experimental animals, for example the ED50 (the dose therapeutically effective in 50% of the population) and LD50 (the dose lethal to 50% of the population).

A “therapeutically effective amount” of either an immunomodulatory agent or a chemotherapeutic agent may range anywhere from 1 to 14 fold or more higher than the typical dose depending upon the patients indication and severity of disease. Accordingly, therapeutically relevant doses of an immunomodulatory agent or a chemotherapeutic agent for any disorder disclosed herein can be, for example, about 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, 125, 150, 175, 200, 225, 250, or 300 fold higher than the prescribed or standard dose. Alternatively, therapeutically relevant doses of an immunomodulatory agent or a chemotherapeutic agent can be, for example, about 1.0×, about 0.9×, 0.8×, 0.7×, 0.6×, 0.5×, 0.4×, 0.3×, 0.2×, 0.1×, 0.09×, 0.08×, 0.07×, 0.06×, 0.05×, 0.04×, 0.03×, 0.02×, or 0.01×.

Disorders for which the sequential dosing regimen may be useful in treating includes one or more of the following disorders: melanoma, prostate cancer, and lung cancer, for example, also include leukemias, including, for example, chronic myeloid leukemia (CML), acute lymphoblastic leukemia, and Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia. Various additional cancers are also included within the scope of protein tyrosine kinase-associated disorders including, for example, the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma. In certain preferred embodiments, the disorder is leukemia, breast cancer, prostate cancer, lung cancer, colon cancer, melanoma, or solid tumors. In certain preferred embodiments, the leukemia is chronic myeloid leukemia (CML), Ph+ ALL, AML, imatinib-resistant CML, imatinib-intolerant CML, accelerated CML, lymphoid blast phase CML.

The terms “cancer”, “cancerous”, or “malignant” refer to or describe the physiological condition in mammals, or other organisms, that is typically characterized by unregulated cell growth. Examples of cancer include, for example, solid tumors, melanoma, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

A “solid tumor” includes, for example, sarcoma, melanoma, colon carcinoma, breast carcinoma, prostate carcinoma, or other solid tumor cancer.

“Leukemia” refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, the present invention provides treatment for chronic myeloid leukemia, acute lymphoblastic leukemia, and/or Philadelphia chromosome positive acute lymphoblastic leukemia (Ph+ ALL).

### Antibodies

The sequential dosing regimen of the present invention may include the use of antibodies as one component of the combination. For example, antibodies that specifically bind to co-stimulatory pathway polypeptides, such as CTLA-4, CD28, CD80, and CD86, preferably Ipilimumab. The term “antibody” is used in the broadest sense and specifically covers monoclonal antibodies, polyclonal antibodies, antibody compositions with polyepitopic specificity, bispecific antibodies, diabodies, chimeric, single-chain, and humanized antibodies, as well as antibody fragments (e.g., Fab, F(ab′)₂, and Fv), so long as they exhibit the desired biological activity. Antibodies can be labeled for use in biological assays (e.g., radioisotope labels, fluorescent labels) to aid in detection of the antibody.

Antibodies that bind to co-stimulatory pathway polypeptides can be prepared using, for example, intact polypeptides or fragments containing small peptides of interest, which can be prepared recombinantly for use as the immunizing antigen. The polypeptide or oligopeptide used to immunize an animal can be derived from the translation of RNA or synthesized chemically, and can be conjugated to a carrier protein, if desired. Commonly used carriers that are chemically coupled to peptides include, for example, bovine serum albumin (BSA), keyhole limpet hemocyanin (KLH), and thyroglobulin. The coupled peptide is then used to immunize the animal (e.g., a mouse, a rat, or a rabbit).

The term “antigenic determinant” refers to that portion of a molecule that makes contact with a particular antibody (i.e., an epitope). When a protein or fragment of a protein is used to immunize a host animal, numerous regions of the protein can induce the production of antibodies that bind specifically to a given region or three-dimensional structure on the protein; each of these regions or structures is referred to as an antigenic determinant An antigenic determinant can compete with the intact antigen (i.e., the immunogen used to elicit the immune response) for binding to an antibody.

The phrase “specifically binds to” refers to a binding reaction that is determinative of the presence of a target in the presence of a heterogeneous population of other biologics. Thus, under designated assay conditions, the specified binding region binds preferentially to a particular target and does not bind in a significant amount to other components present in a test sample. Specific binding to a target under such conditions can require a binding moiety that is selected for its specificity for a particular target. A variety of assay formats can be used to select binding regions that are specifically reactive with a particular analyte. Typically a specific or selective reaction will be at least twice background signal or noise and more typically more than 10 times background. For purposes of the present invention, compounds, for example small molecules, can be considered for their ability to specifically bind to co-stimulatory pathway polypeptides described herein.

### Kits

For use in the diagnostic and therapeutic applications described or suggested above, kits are also provided by the invention. Such kits can, for example, comprise a carrier means being compartmentalized to receive in close confinement one or more container means such as vials, tubes, and the like, each of the container means comprising one of the separate elements to be used in the method. For example, one of the container means can comprise a means for performing an absolute lymphocyte count on a patient sample and/or instructions for interpreting the ALC value obtained. Another example of a container means can comprise one or more vials containing a pharmaceutically acceptable amount of a co-stimulatory pathway modulator.

The kit of the invention will typically comprise the container described above and one or more other containers comprising materials desirable from a commercial and user standpoint, including buffers, diluents, filters, needles, syringes, and package inserts with instructions for use. A label can be present on the container to indicate that the composition is used for a specific therapy or non-therapeutic application, and can also indicate directions for either in vivo or in vitro use, such as those described above.

Kits useful in practicing therapeutic methods disclosed herein can also contain a compound that is capable of inhibiting the co-stimulatory pathway. Specifically contemplated by the invention is a kit comprising an anti-CTLA-4 antibody, either alone or in combination with another immunotherapy agent, such as PROVENGE®; a tubulin stabilizing agent (e.g., pacitaxol, epothilone, taxane, etc.); and/or a second co-stimulatory pathway modulator, such as, tremelimumab. In addition, contemplated by the invention is a kit comprising an increased dose and/or dosing frequency regimen of a co-stimulatory pathway modulator, and any other combination or dosing regimen comprising a tubulin stabilizing agent (e.g., pacitaxol, epothilone, taxane, etc.); and/or a second co-stimulatory pathway modulator, such as, tremelimumab.

In addition, the kits can include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

The kit can also comprise, for example, a means for obtaining a biological sample from an individual. Means for obtaining biological samples from individuals are well known in the art, e.g., catheters, syringes, and the like, and are not discussed herein in detail.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

The following representative examples contain important additional information, exemplification and guidance which can be adapted to the practice of this invention in its various embodiments and the equivalents thereof. These examples are intended to help illustrate the invention, and are not intended to, nor should they be construed to, limit its scope.

### REFERENCES

1. Clemente C. G. et al., “Prognostic value of tumor infiltrating lymphocytes in the vertical growth phase of primary cutaneous melanoma”, Cancer, 77(7):1303-1310 (1996).
2. Kirkwood, J. M. et al., “Studies of interferons in the therapy of melanoma”, Semin. Oncol., 18(5) (Suppl. 7):83-90 (1991).
3. Kirkwood, J. M. et al., “High- and low-dose interferon alfa-2b in high-risk melanoma: first analysis of intergroup trial E1690/S9111/C9190”, J. Clin. Oncol., 22(6):1118-1125 (Mar. 15, 2004).
4. Lenschow, D. J. et al., “CD28/B7 system of T cell costimulation”, Ann Rev. Immunol., 14:233-258 (1996).
5. Schwartz, R. H., “Costimulation of T lymphocytes: the role of CD28, CTLA4, and B7/BB1 in interleukin-2 production and immunotherapy”, Cell, 71(7):1065-1068 (1992).
6. Chen, L. S. et al., “Costimulation of antitumor immunity by the B7 counterreceptor for the T lymphocyte molecules CD28 and CTLA-4”, Cell, 71(7):1093-1102 (1992).
7. Townsend, S. E. et al., “Tumor rejection after direct costimulation of CD8+ T cells by B7-transfected melanoma cells”, Science, 259(5093):368-370 (1993).
8. Townsend, S. E. et al., “Specificity and longevity of antitumor immune responses induced by B7-transfected tumors”, Cancer Res., 54(24):6477-6483 (1994).
9. Allison, J. P. et al., “Manipulation of costimulatory signals to enhance antitumor t cell responses”, Curr. Opin. Immunol., 7(5):682-686 (1995).
10. Linsley, P. S. et al., “CTLA-4 is a second receptor for the B cell activation antigen B7”, J. Exp. Med., 174(3):561-569 (1991).
11. Thompson, C. B. et al., “The emerging role of CTLA-4 as an immune attenuator”, Immunity, 7(4):445-450 (1997).
12. Walunas, T. L. et al., “CTLA-4 can function as a negative regulator of T cell activation”, Immunity, 1(5):405-413 (1994).
13. Kearney, E. R. et al., “Antigen-dependent clonal expansion of a trace population of antigen-specific CD4+ T cells in vivo is dependent on CD28 costimulation and inhibited by CTLA-4”, J. Immunol., 155(3):1032-1036 (1995).
14. Krummel, M. F. et al., “CD28 and CTLA-4 have opposing effects on the response of T cells to stimulation”, J. Exp. Med., 182(2):459-465 (1995).
15. Krummel, M. F. et al., “Superantigen responses and co-stimulation: CD28 and CTLA-4 have opposing effects on T cell expansion in vitro and in vivo”, Int. Immunol., 8(4):519-523 (1996).
16. Tivol, E. A. et al., “Loss of CTLA-4 leads to massive lymphoproliferation and fatal multiorgan tissue destruction, revealing a critical negative regulatory role of CTLA-4”, Immunity, 3(5):541-547 (1995).
17. Waterhouse P. et al., “Lymphoproliferative disorders with early lethality in mice deficient in CTLA-4”, Science, 270(5238):985-988 (1995).
18. Chambers C. A. et al., “Lymphoproliferation in CTLA-4-deficient mice is mediated by costimulation-dependent activation of CD4+ T cells”, Immunity, 7(6):885-895 (1997).
19. MDX-010, Ipilimumab Investigators' brochure. Version: Edition 9. Dated: 5-Sep.-2006.
20. Sandler, A. et al., “Paclitaxel-Carboplatin Alone or with Bevacizumab for Non-Small-Cell Lung Cancer”, New Engl. J. Med., 355(24):2542-2550 (Dec. 14, 2006).
21. Gridelli, C. et al., “Carboplatin plus paclitaxel in extensive small cell lung cancer: a multicentre phase 2 study”, Br. J. Cancer, 84(1):38-41 (Jan. 5, 2001).
22. Vansteenkiste, J., “Multi-center, double-blind, randomized, placebo-controlled phase II study to assess the efficacy of recombinant MAGE-A3 vaccine as adjuvant therapy in stage IB/II MAGE-A3-positive, completely resected, non-small cell lung cancer (NSCLC)”, Presented at American Society of Oncology Annual Meeting, 2006.
23. Murray, N., “A liposomal MUC1 vaccine for treatment of non-small cell lung cancer (NSCLC); updated survival results from patients with stage IIIB disease”, Presented at American Society of Oncology Annual Meeting, 2005.
24. Wang, D., “Antitumor activity of a synthetic agonist of TLR9 in preclinical lung cancer models”, Presented at American Society of Oncology Annual Meeting, 2006.
25. Rigas, J., “Taxane-Platinum Combinations in Advanced Non-Small Cell Lung Cancer: A Review”, The Oncologist, 9 (Suppl. 2):16-23 (2004).
26. Schmittel, A. et al., “A randomized phase II trial of irinotecan plus carboplatin versus etoposide plus carboplatin treatment in patients with extended disease small-cell lung cancer”, Annals of Oncology, 17(4):6663-6667 (April 2006).
27. TAXOL® (paclitaxel) US full prescribing information available at Bristol-Myers Squibb Company corporate website.
27. PARAPLATIN® (carboplatin) US full prescribing information available at Bristol-Myers Squibb Company corporate website.
28. Pocock, S. J. et al., “Sequential treatment assignment with balancing for prognostic factors in the controlled clinical trial”, Biometrics, 31:103-115 (1975).
29. Brookmeyer, R. et al., “A confidence interval for the median survival time”, Biometrics, 38:29-41 (1982).
30. Clopper, C. J. et al., :The use of confidence or fiducial limits illustrated in the case of the binomial”, Biometrika, 26:404-413 (1934).
31. Wei, L. J. et al., “Two-sample asymptotically distribution-free tests for incomplete multivariate observations”, J. Am. Stat. Assoc., 79:653-661 (1984).
32. Gabriel et al., Clin. Cancer Res., 13:785-788 (2007).
33. Wolchok et al., Lancet Oncol., 11(2):155-164 (2010).
34. Ku et al., Cancer, 116(7):1767-1775 (2010).
35. West et al., Clin. Lung Cancer., 10(Suppl. 1):S41-S46 (2009).
36. Peled et al., Future Med., 1(1):19-25 (2009).
37. Gerard et al., Mol. Oncol., 3(5-6):409-424 (2009).
38. Wolchok et al., Clin. Cancer Res., 15(23):7412-7420 (2009).
39. Harmankaya et al., World Meeting of Melanoma/Skin Cancer Centers, Berlin, Germany, Poster #37.
40. Hodi, F. S. et al., N. Engl. J. Med., 363(8):711-723 (August 2010).
41. O'Day, S. J. et al., Ann. Oncol., 10 (February 2010).
42. Urba, W. J. et al., J. Clin. Oncol., 26 (May 20 Suppl.), Abstract No. 3018 (2008).
43. Slovin, S. F. et al., J. Clin. Oncol., 27:15S (Suppl.) Abstract No. 5138, (2009).
44. Yang, J. C. et al., J. Immunother., 30(8):825-830 (November-December 2007).
45. Royal, R .E. et al., GI Canc. Symp., Abstract No. 153 (2009).
46. Wolchok, J. D. et al., Lancet Oncol., 11(2):155-164 (February 2010).
47. Zhang, L. et al., “Differential impairment of regulatory T cells rather than effector T cells by paclitaxel-based chemotherapy”, Clin. Immunol., 129:219-229 (2008).
48. Kodumudi, K. N. et al., Clin. Cancer Res., 16:4583-4594 (2010).
49. Ferretti, S. et al., Clin. Cancer Res., 11:7773-7784 (2005).

### EXAMPLES

### Example 1

### Methods for Comparing Therapeutic Efficacy of Concurrent Versus Sequential Dosing Regimens with Immunotherapeutic and Chemotherapeutic Agents in a Phase II Clinical Study

A phase IIb, randomized, double-blind, parallel, three arm, multicenter clinical trial, protocol CA184041, was begun to evaluate the efficacy and safety of Ipilimumab (BMS-734016) in combination with the chemotherapeutic agents TAXOL®/PARAPLATIN® (Paclitaxel/Carboplatin) compared to TAXOL®/PARAPLATIN® alone in previously untreated subjects with lung cancer. The '041 study included 80-100 patients.

The primary objective of the study was to compare the immune-related progression free survival (irPFS) of subjects receiving Ipilimumab in combination with either concurrent TAXOL®/PARAPLATIN® (“concurrent”; Arm A) or TAXOL®/PARAPLATIN® (“sequential”; Arm B) to that of subjects receiving TAXOL®/PARAPLATIN® alone (Arm C) in Stage IIIb/IV NSCLC subjects using irRC as per the assessment of an independent review committee (IRC).

Several secondary objectives included comparing PFS for the NSCLC subjects in Arm A vs. Arm C and Arm B vs Arm C using mWHO; comparing the irPFS and PFS for extensive SCLC subjects in Arm A vs. Arm C and Arm B vs Arm C using the irRC and mWHO, respectively; comparing overall survival in Arm A vs Arm C and Arm B vs Arm C in subjects with NSCLC and in subjects with SCLC; comparing immune-related best overall response rate (irBORR), immune-related disease control rate (irDCR), best overall response rate (BORR), disease control rate (DCR) of Arm A vs Arm C and Arm B vs Arm C using irRC and mWHO, respectively, for subjects with NSCLC and for subjects with SCLC; evaluating the safety profile in each arm for subjects with NSCLC and for subjects with SCLC; and evaluating the association between safety and efficacy in subjects with NSCLC and in subjects with SCLC.

### Methods

Study Design: The '041 trial was a double-blind, randomized, parallel, three arm, multicenter, Phase II, study in previously untreated subjects with lung cancer to evaluate the efficacy and safety of two schedules of Ipilimumab (10 mg/kg) in combination with TAXOL® (175 mg/m2) and PARAPLATIN® (AUC=6) (up to 6 doses) compared to subjects receiving TAXOL®/PARAPLATIN® chemotherapy alone at the same doses.

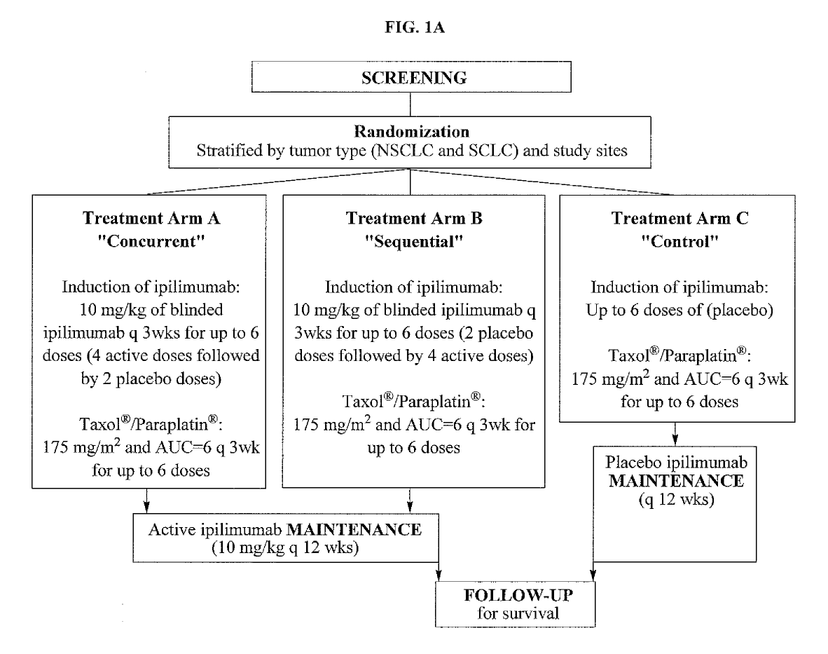

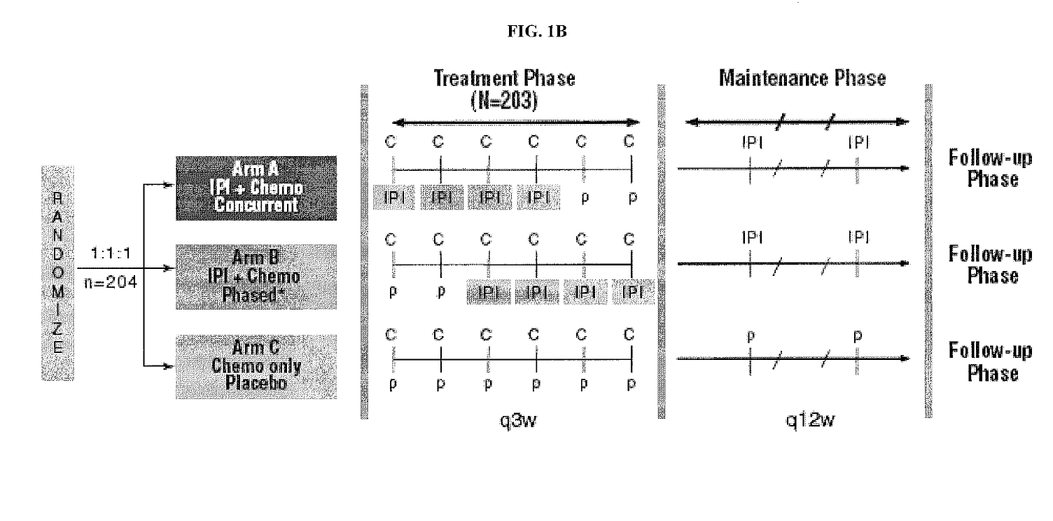

Approximately 210 NSCLC subjects and 120-210 SCLC subjects will be randomized (1:1:1) and stratified by tumor type and study site to one of three possible double blind treatment regimens. Each arm of the study design is summarized below and illustrated schematically in FIGS. 1A-B.
Arm A (Concurrent): Six doses of blinded Ipilimumab dosed with TAXOL®/PARAPLATIN®. The first 4 doses must be active Ipilimumab followed by TAXOL®/PARAPLATIN® and the last 2 doses must be placebo Ipilimumab followed by TAXOL®/PARAPLATIN®. A maximum of 6 doses of chemotherapy will be permitted.
Arm B (Sequential): Six doses of blinded Ipilimumab dosed with TAXOL®/PARAPLATIN®. The first 2 doses must be placebo Ipilimumab followed by TAXOL®/PARAPLATIN® and the last 4 doses must be active Ipilimumab followed by TAXOL®/PARAPLATIN®. A maximum of 6 doses of chemotherapy will be permitted.
Arm C (Control): Six doses of placebo Ipilimumab dosed with six doses of TAXOL®/PARAPLATIN®.

Number of Subjects per Group: 210 NSCLC subjects and 120-210 SCLC subjects were randomized into Arm A, Arm B, or Arm C in a 1:1:1 ratio.

Study Population: Men and women who are ≦18 years old with histologically or cytologically confirmed lung cancer (Stage IIIb/IV NSCLC or extensive stage SCLC) with ECOG performance ≦1, who have met screening laboratory requirements, and who are previously untreated. Subjects with specific underlying autoimmune diseases (particularly gastrointestinal) or paraneoplastic syndromes related to SCLC were excluded.

Demographics of the target patient population are outlined in Table 1.

TABLE 1
Demography and Patient Characteristics -
Randomized NSCLC Subjects

| | Arm A | Arm B | Arm C | |
|---|---|---|---|---|
| | Concurrent | Phased | Placebo | Total |
| | IPI + Chemo | IPI + Chemo | Chemo Only | (N = |
| | (N = 70) | (N = 68) | (N = 66) | 204) |
| Mean Age (years) | 60.3 | 60.6 | 60.6 | 60.5 |
| Gender (%) | | | | |
| Male | 76 | 72 | 74 | 74 |
| Female | 24 | 28 | 26 | 26 |
| Disease Stage (%)^{†} | | | | |
| IIIb | 19 | 10 | 40 | 21 |
| IV | 81 | 90 | 60 | 79 |
| Cell Type (%)* | | | | |
| Adenocarcinoma | 50 | 44 | 58 | 51 |
| Squamous Cell | 30 | 31 | 23 | 28 |
| Carcinoma | | | | |
| Large Cell | 9 | 16 | 11 | 12 |
| Carcinoma | | | | |
| Broncho-Alveolar | 1 | 2 | 0 | 1 |
| Carcinoma | | | | |
| Other | 9 | 6 | 5 | 6 |
| Unknown | 1 | 2 | 5 | 3 |
| ECOG PS (%)^{†} | | | | |
| 0 | 27 | 37 | 23 | 29 |
| 1 | 73 | 63 | 77 | 71 |

*Due to percent rounding, the cell type percentages do not always total 100%
^{†}Potential numerical imbalance in ECOG and disease stage.

Investigational Product(s), Dose and Mode of Administration, Duration of Treatment with Investigational Product(s):

Blinded Study Drug: Ipilimumab 10 mg/kg or matched placebo administered as a single dose intravenously over 90 minutes every 3 weeks (up to 6 doses) as part of induction. Subjects may receive additional maintenance Ipilimumab/placebo at a dose of 10 mg/kg and administered intravenously over 90 minutes every 12 weeks starting 24 weeks after the first Ipilimumab/placebo dose. Dose reductions were not permitted. TAXOL®: 175 mg/m2 administered as a single dose intravenously over 3 hours every 3 weeks (up to 6 doses). Dose modifications (reductions as well as delays) are as per product label. PARAPLATIN®: AUC=6 administered as a single dose intravenously over 30 minutes every 3 weeks (up to 6 doses). Dose modifications (reductions as well as delays) are as per product label. Treatment with blinded Ipilimumab (active or placebo) proceeded until immune-related tumor progression as defined by the irRC is observed or intolerable toxicity occurs.

Subjects who were thought by the Investigator to be experiencing clinical benefit but are discontinued from blinded study drug due to toxicity will continue with Maintenance Phase TAs and study procedures until they move onto an alternative systemic anti-cancer therapy or withdrawal consent.

Treatment with TAXOL® and PARAPLATIN® proceeded until immune-related tumor progression, as defined by the irRC, reached a maximum of 6 treatment doses, unacceptable toxicity thought to be related to any study drug, pregnancy, or withdrawal of consent occurred.

### Study Assessments and Primary Endpoints:

Tumor Assessments (TA): To ensure a uniform TA schedule, radiological imaging (e.g., MRI/CT of brain, bone, chest, abdomen, pelvis and other soft tissue as applicable) was performed for all subjects at screening and every 6 weeks while in Treatment and every 12 weeks while in Maintenance. For subjects who moved into Follow-Up, formal TAs are no longer required.

Efficacy: The irRC represent further modifications of the mWHO criteria reflecting the clinical experience with Ipilimumab in over 20 completed and/or ongoing clinical studies in which objective and durable responses (as per mWHO) were observed in subjects following progression and without intervening alternative anti-cancer therapy. As such, the irRC was designed to capture clinical activity of

Ipilimumab immunotherapy that may not be adequately addressed by the mWHO criteria. Final assessment of tumor response-related parameters such as irPFS and response are assessed by the IRC using irRC. The irRC, as per Investigator assessment, guided clinical care (i.e., duration of dosing) during the course of the study. mWHO and irRC criteria are summarized below for comparison.

| Response | mWHO* | irRC^{#} |
|---|---|---|
| Complete | All lesions gone | All index + new |
| Response | | lesions gone |
| Partial | SPD of index lesions | SPD of Index + new |
| Response | decreases >50% new lesions | lesions decreases >50% |
| | not allowed | new lesions measured |
| Stable | SPD of index lesions neither | SPD of index + new |
| Disease | CR, PR or PD new lesions | lesions neither irCR, |
| | not allowed | irPR, nor irPD new |
| | | lesions measured |
| Progressive | SPD of index lesions | SPD of index + new |
| Disease | increases >25% OR new | lesions increases >25%** |
| | lesions OR unequivocal | |
| | progression of non-index | |
| | lesions | |

SPD = sum of products of perpendicular diameters;
*includes index and non-index lesions;
**Progression to be confirmed with 2 consecutive assessments.
^{#}includes index (measurable lesions) only;

The primary endpoint is progression free survival using irRC (as per the IRC assessment) in NSCLC.

Secondary endpoints included overall survival (OS) and response-related endpoints such as disease control rate (DCR), best overall response rate (BORR), duration of response using both the mWHO (as per IRC TA) and irRC as per both IRC and the investigator.

### Summary of Statistical Analyses:

The primary objectives were to compare irPFS in subjects receiving Ipilimumab in combination with chemotherapy administered concurrently (Arm A) or sequentially (Arm B), with chemotherapy alone (Arm C) in NSCLC subjects using irRC per IRC TA. The primary efficacy analyses was based on all randomized subjects.

irPFS, PFS, OS, duration of response, immune-related duration of response was calculated by treatment arm for each tumor type. The survival probabilities of irPFS, PFS, OS, duration of response, and immune-related duration of response were estimated using Kaplan-Meier (KM) product limit method, medians with corresponding two-sided 80% confidence intervals and reported using the method of Brookmeyer and Crowley. KM curves were also plotted by treatment arm for each tumor type. For each tumor type, hazard ratios and the corresponding two-sided 80% confidence intervals (CI) were constructed for Arm A vs Arm C and for Arm B vs Arm C for irPFS, PFS and OS. For each tumor type, the log-rank test with one-sided alpha of 0.1 was performed to compare irPFS, PFS, and OS in Arm A vs Arm C and in Arm B vs Arm C. No alpha adjustment for multiple comparison was planned.

BORR, Disease control rate, irBORR and immune-related disease control rate were estimated by treatment arm for each tumor type. An exact two-sided 80% CI in each arm was computed for the above rates using the method of Clopper and Pearson. For each tumor type, the differences of the above rates for Arm A vs Arm C and for Arm B vs Arm C and corresponding two-sided 80% confidence intervals was computed respectively.

For each tumor type, the summary tables were tabulated to evaluate the differences of response related endpoints between mWHO and irRC as per IRC TA by treatment group.

The primary PFS analysis was performed when a total of approximately 150 irPFS events as per irRC was observed among three treatment arms in NSCLC subjects. At the time of primary irPFS analysis in NSCLC subjects, other efficacy analyses were also performed.

Demographic and baseline characteristics were summarized by treatment group for each tumor type using descriptive statistics for all randomized subjects.

Safety was summarized and listed for all treated subjects using the National Cancer Institute (NCI) Common Terminology Criteria for Adverse Events (CTCAE) version 3.0 by treatment arm for each tumor type. All treatment emergent AEs, drug-related treatment emergent AEs, serious AEs and drug-related SAEs were tabulated using worst grade per NCI CTCAE v3.0 criteria by system, organ, class, and preferred term. The listings by subject were produced for all deaths, all SAEs and all AEs leading to discontinuation of study drug. On-study laboratory parameters including hematology, coagulation, chemistry, liver function and renal function were summarized using worst grade per NCI CTCAE v3.0 criteria.

### RESULTS

### Interim Data Analysis of the CA184041 Study

CA184041 is an ongoing randomized study wherein patients with previously untreated Non-Small Cell Lung Cancer (NSCLC) as well Extensive Disease Small-Cell Lung cancer (ED-SCLC) were randomized to be treated with carboplatin and paclitaxel plus Ipilimumab (using two different administration schedules) or a placebo. This topline data set presents results for NSCLC subjects only. ED SCLC data are still maturing.

Patients enter a treatment phase where they receive up to 6 doses of paclitaxel (175 mg/m2) and carboplatin (AUC=6), and are randomized to receive one of three concomitant treatments: 4 doses of 10 mg/kg Ipilimumab followed by two doses of placebo (Arm A, concurrent regimen); 2 doses of placebo followed by 4 doses of 10 mg/kg Ipilimumab (Arm B, sequential regimen); or 6 doses of placebo (Arm C, placebo regimen). Randomization was stratified by cancer diagnosis (NSCLC vs. ED-SCLC). Following the end of the treatment phase, subjects experiencing anti-cancer benefit and tolerating treatment enter a Maintenance Phase where they receive randomized treatment (10 mg/kg Ipilimumab for concurrent and consecutive regimens, placebo for placebo regimen) q12 weeks.

CA184041 is a proof-of-concept efficacy trial with co-primary endpoints to compare immune-related progression-free survival (irPFS) between the concurrent regimen and the placebo regimen, and between the sequential regimen and the placebo regimen, respectively, in Stage IIIb or IV NSCLC patients. Study results will be used to assess the feasibility of and guide the treatment regimen to be used in a Phase III program for Ipilimumab in advanced lung cancer.

The primary endpoint analysis for irPFS is final for this cohort and all other endpoint analyses are intermediate. All SCLC results and subsequent NSCLC results beyond irPFS will be presented at the completion of the study. Cohorts are defined based on the Investigator baseline CRF

Response-related efficacy assessments for the purpose of guiding patient-management decisions were made by investigators using immune-related response criteria (irRC). An Independent Review Committee (IRC) reviewed all imaging data and assessed tumor response separately using both irRC criteria and modified World Health Organization (mWHO) criteria. Tumor imaging was performed every 6 weeks during the Treatment Phase and every 12 weeks during the Maintenance Phase. Subjects withdrawing from treatment were asked to continue tumor assessment imaging. The primary endpoint of irPFS is based on irRC criteria and IRC review.

As used in this study, immune-related response criteria are based on the sum of objectively measurable tumor volume including both index and measurable new lesions. New lesions did not constitute progression unless the total tumor volume exceeded 25% above nadir. Although investigators were encouraged to confirm irPD prior to treatment withdrawal, confirmation was not considered in the efficacy analysis. mWHO criteria considered the presence of any new lesion, or unequivocal progression of non-index lesions, as progression. By both criteria, progression could be declared at any assessment and irSD began at Week 7.

This report provides a final analysis of the primary efficacy endpoint of irPFS per IRC in NSCLC subjects. It also provides intermediate analyses of PFS per IRC, irPFS per investigator, OS, duration of response, immune-related best overall response rate (irBORR) per IRC, immune-related disease control rate (irDCR) per IRC; safety data tabulating AEs and irAEs; exposure data on number of doses; and drug discontinuation data.

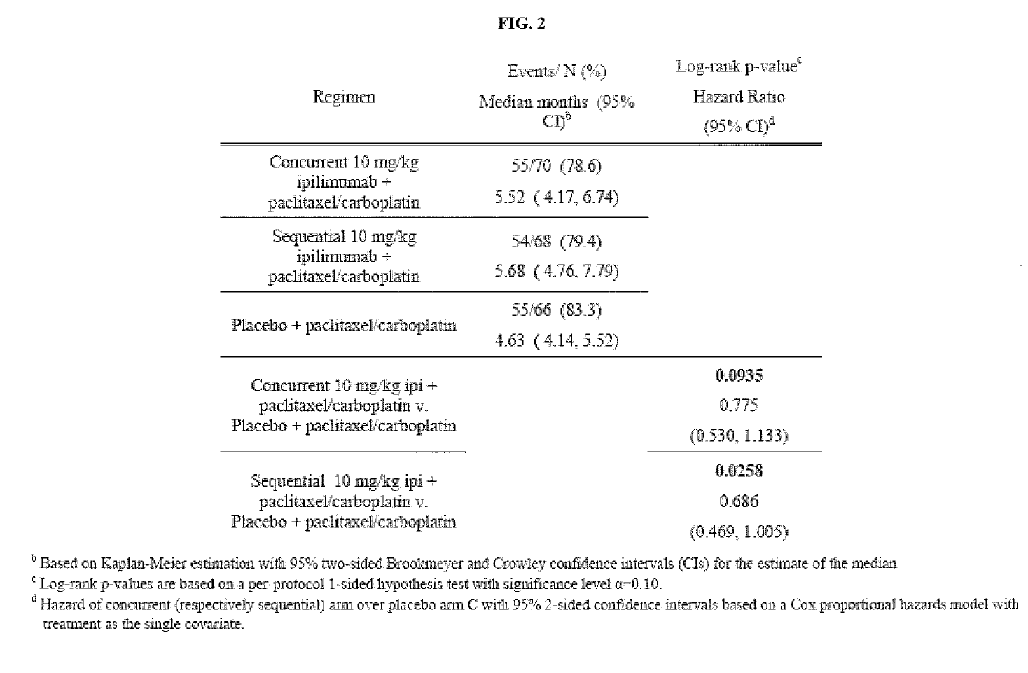

As discussed herein, the primary objective of this study was to compare immune-related progression free survival (irPFSa) between subjects receiving paclitaxel/carboplatin in combination with each of two schedules of Ipilimumab (concurrent or sequential schedule, respectively) and subjects receiving paclitaxel/carboplatin in combination with placebo in Stage IIb/IV NSCLC patients. As shown in FIG. 2, Both Ipilimumab regimens were superior to placebo under protocol-defined Phase II significance criteria (one-sided test with a=0.10). Improvement in irPFS was numerically greater in the sequential arm but influence of imbalance in baseline patients characteristics cannot be ruled out. More early progression (within 6 weeks after randomization) occurred in the concomitant arm than in the placebo arm.

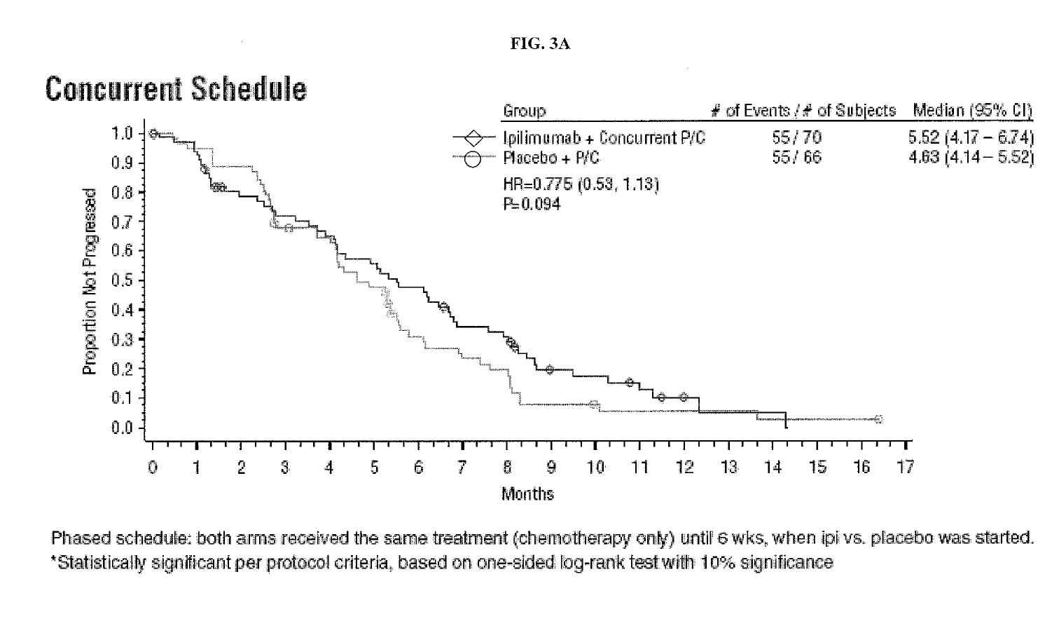

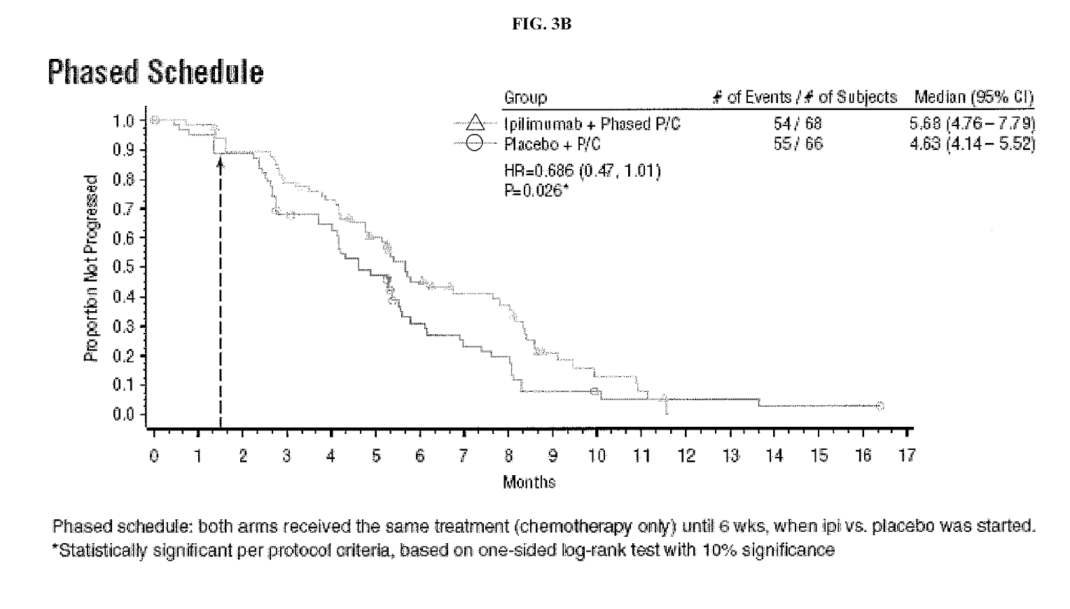

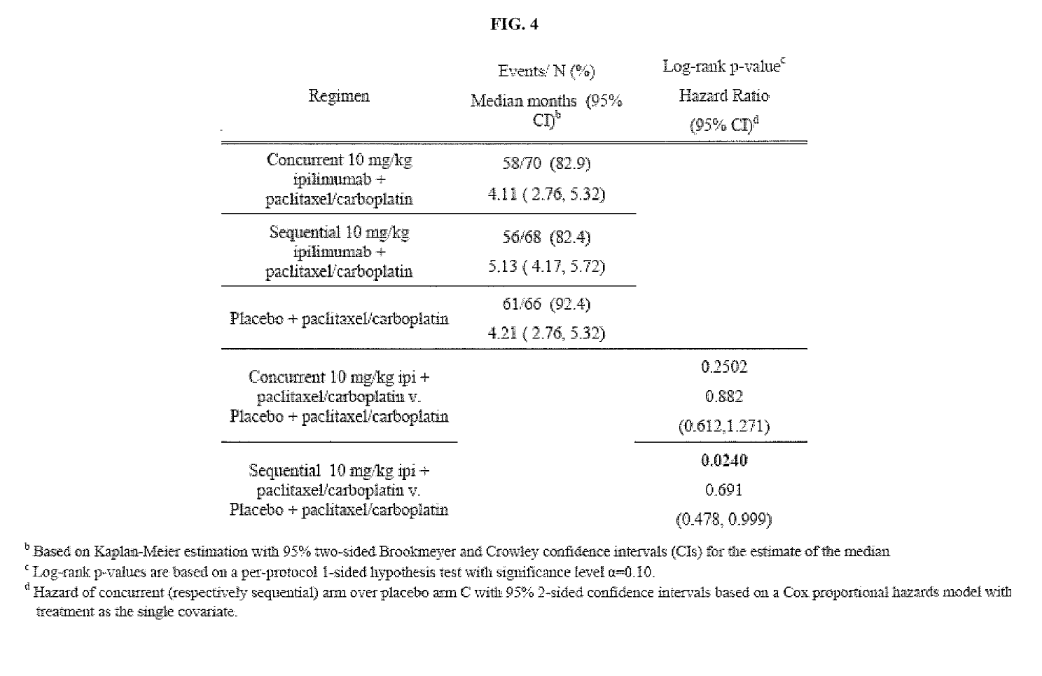

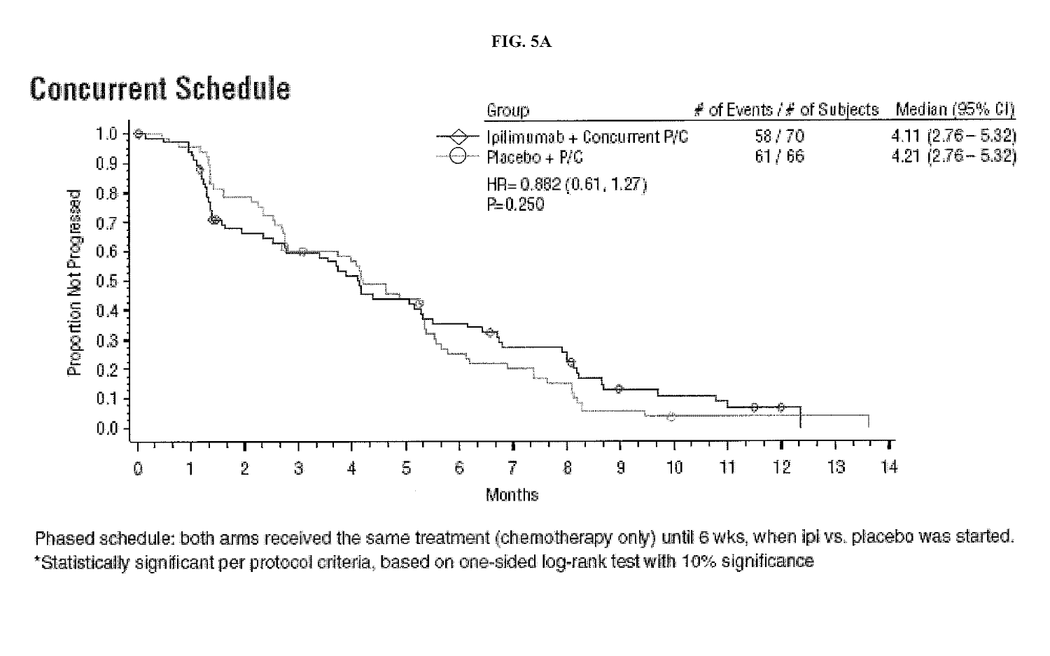

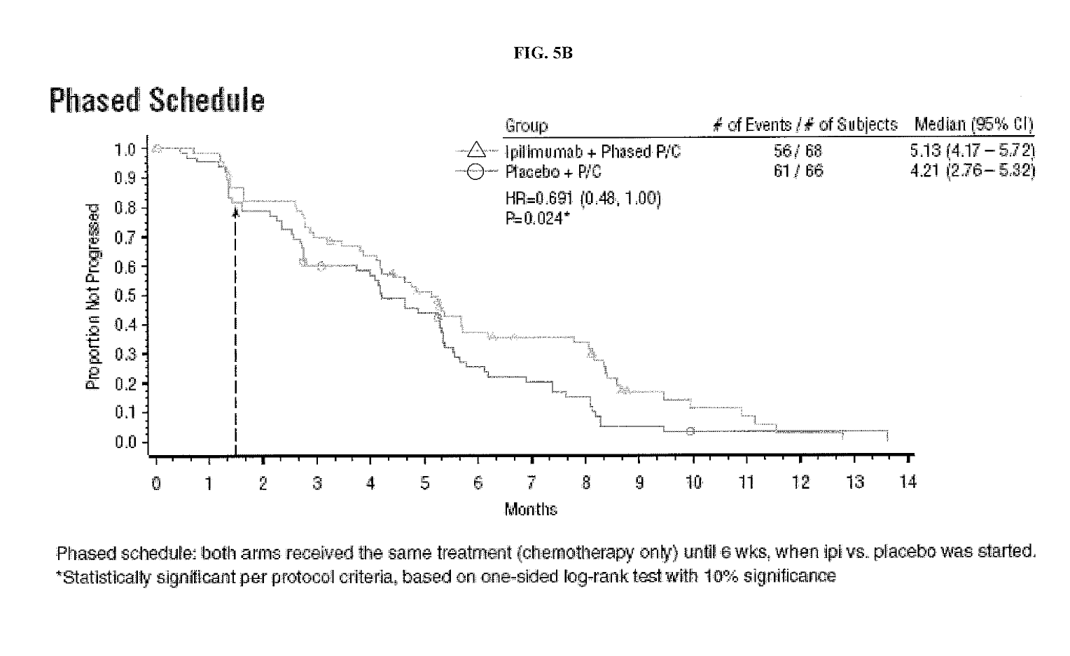

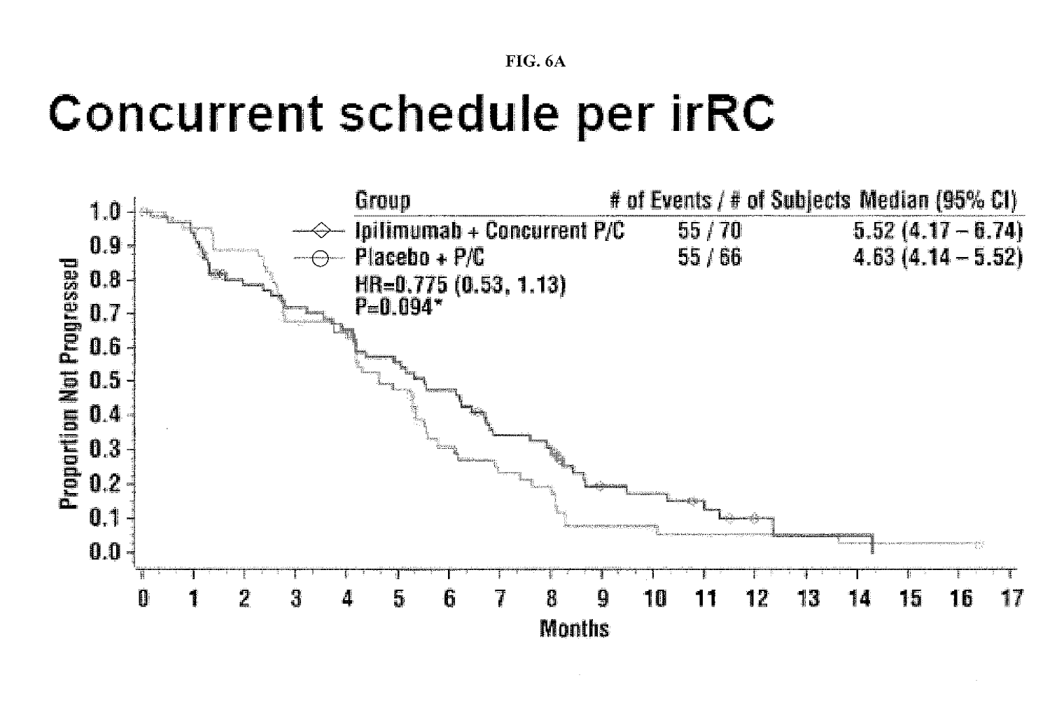

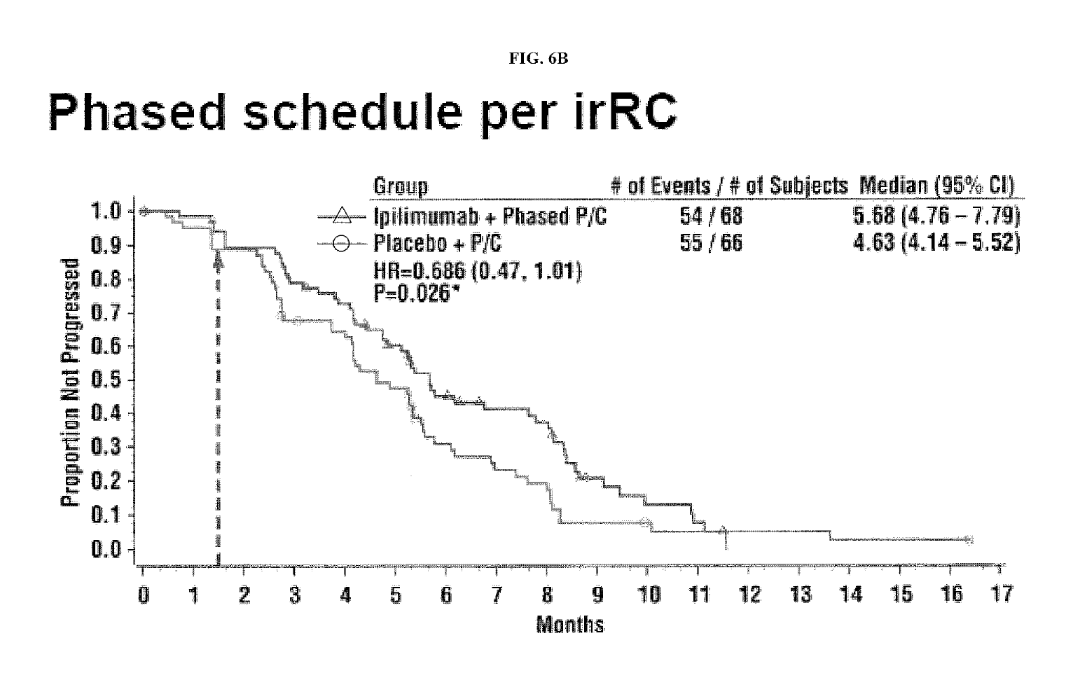

A secondary objective of the study was to compare progression free survival (PFSa) between the concurrent (respectively sequential) and placebo regimens. As shown in FIG. 3, only the sequential regimen showed statistically significant efficacy v. placebo based upon this preliminary analysis.

Another secondary objective of the study was to compare overall survival (OS) between the concurrent (respectively sequential) and placebo regimens. At this stage of the analysis, there was no statistically significant result (data not shown). The analysis was immature. The survival outcome in the control arm was consistent with protocol assumptions. Only 119 deaths have been observed. The sequential arm appeared numerically encouraging but statistical significance was not met. The final analysis will be performed at study closure. Subsequently, deaths will be recorded up to two years after LPFV.

Another secondary objective of the study was to compare immune-related best overall response (irBORR), immune-related disease control rate (irDCR), and duration of immune-related response between the concurrent (respectively sequential) and placebo regimens. Re: irBORR rate, preliminary analysis suggested the irBORR rate appeared to be numerically better in the sequential arm (data not shown).

Numerical rates were consistent with the low range of historical data (assuming irBORR and mWHO rates comparable.) Re: irDCR, preliminary analysis suggested irDCR appeared similar across regimens (data not shown). There is a possibility that the concurrent regimen has a numerically worse outcome than the control.

Another secondary objective of the study was to compare immune-related best overall response (irBORR), immune-related disease control (irDC), and duration of immune-related response between the concurrent (respectively sequential) and placebo regimens. Preliminary analysis at the time of database lock suggested the concurrent arm appeared to have the longest duration of response, followed by the sequential arm and placebo arm (data not shown). The patients who were censored were still on study. Additional follow-up will be performed.

Another secondary objective of the study was to evaluate the safety profile in both the concurrent and sequential arms of the study. Regarding adverse events, preliminary analysis showed that almost all subjects had an adverse event regardless of causality (data not shown). 57.8% of the concurrent-arm, 51.2% of the sequential-arm and 41.4% of the control-arm subjects had a high-grade (3/4) AE. Grade 5 AEs were balanced across arms and most were disease progression. Regarding, immune related adverse events, preliminary analysis showed that most reported irAEs were low-grade (1/2). irAE rates in the control arm were numerically 10 to 15% lower compared to the Ipilimumab arms, however, all irAEs in the control arm are characteristic chemotherapy events (data not shown). There was no substantial new toxicity in the Ipilimumab arms as a consequence of the drug combination. Drug-related deaths were rare.

Another secondary objective of the study was to evaluate the safety profile in terms of exposure endpoints in both the concurrent and sequential arms of the study. Regarding safety in terms of the exposure endpoints for Ipilimumab, the number of doses of Ipilimumab per subject at the time of the intermediate analysis was as follows: 53.6% of concurrent subjects and 31.3% of sequential subjects completed at least 4 doses of Ipilimumab. This may represent potential differences in tolerability between arms but also the discontinuation due to disease progression, which may be declared earlier in the sequential arm relative to the number of doses received, probably as a consequence of initial tumor flare induced by intra-tumor inflammation. The latter is supported by approximately 12% of subjects having not received any Ipilimumab in the sequential arm (likely due to discontinuation due to early disease progression). Regarding safety in terms of exposure endpoints for paclitaxel at the time of the intermediate analysis, the number of doses of paclitaxel per subject were as follows: 46.5% of concurrent subjects, 65.7% of sequential subjects, and 53.8% of placebo subjects received at least 5 doses of paclitaxel. The dosing pattern is generally consistent with ir-progression pattern. Regarding safety in terms of exposure endpoints for carboplatin at the time of the intermediate analysis, the number of doses of carboplatin per subject was as follows: 47.9% of concurrent subjects, 62.7% of sequential subjects, and 56.9% of placebo subjects received at least 5 doses of carboplatin. The dosing pattern is generally consistent with ir-progression pattern. The higher number of patients who received only 1-2 doses of carboplatin might be linked to a higher proportion of early progressors in the concurrent arm. This is currently being investigated.

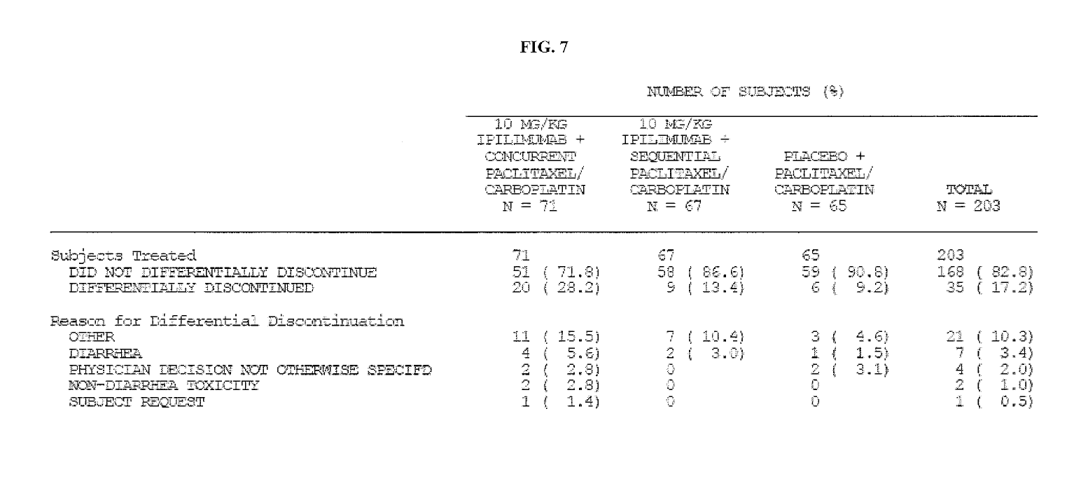

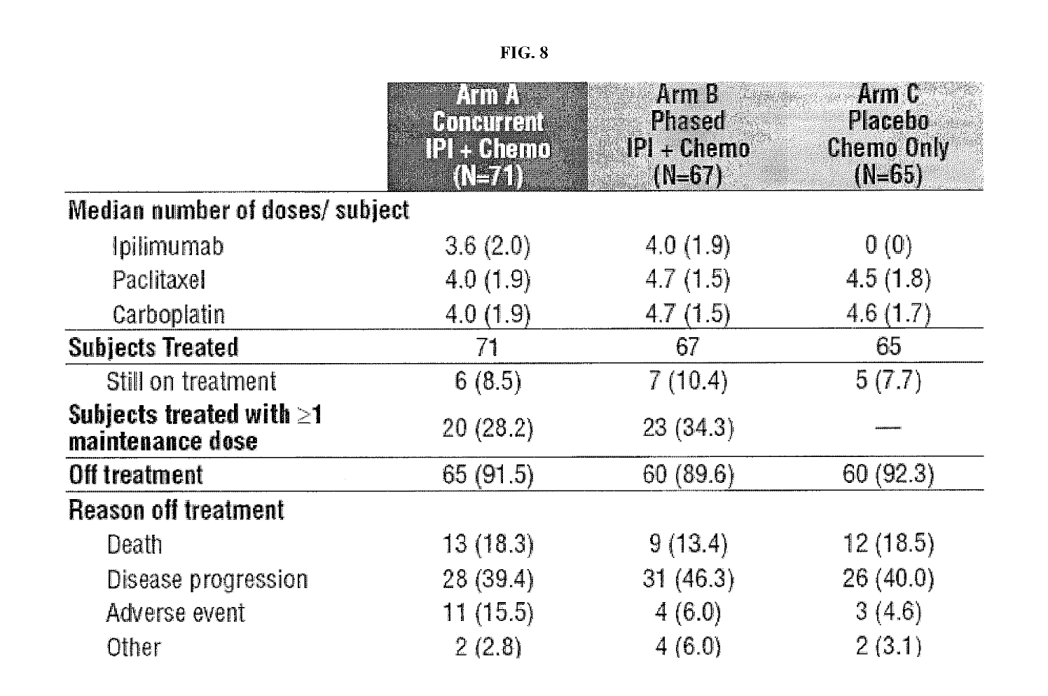

Another secondary objective of the study was to evaluate the discontinuation rates for each arm. As shown in FIG. 7, subjects in the concurrent arm differentially discontinued Ipilimumab/placebo (separately from other study drugs) at a numerically higher rate than sequential or placebo arms. As shown in FIG. 8, reasons for final discontinuation of all study drugs were similar across treatment arms, with more concurrent arm patients withdrawing due to adverse events.

### Final Data Analysis of the CA184041 Study

Once database lock had been achieved after the conclusion of the CA184041 randomized, double-blind, parallel, three arm, multicenter, phase II trial evaluating the efficacy and safety of ipilimumab (BMS-734016) in combination with paclitaxel/carboplatin compared to placebo in combination with paclitaxel/carboplatin in previously untreated subjects with Stage IIIb/IV non-small cell lung cancer (NSCLC) and in previously untreated subjects with extensive-stage disease small-cell lung cancer (ED-SCLC), a final analysis of the data was performed.

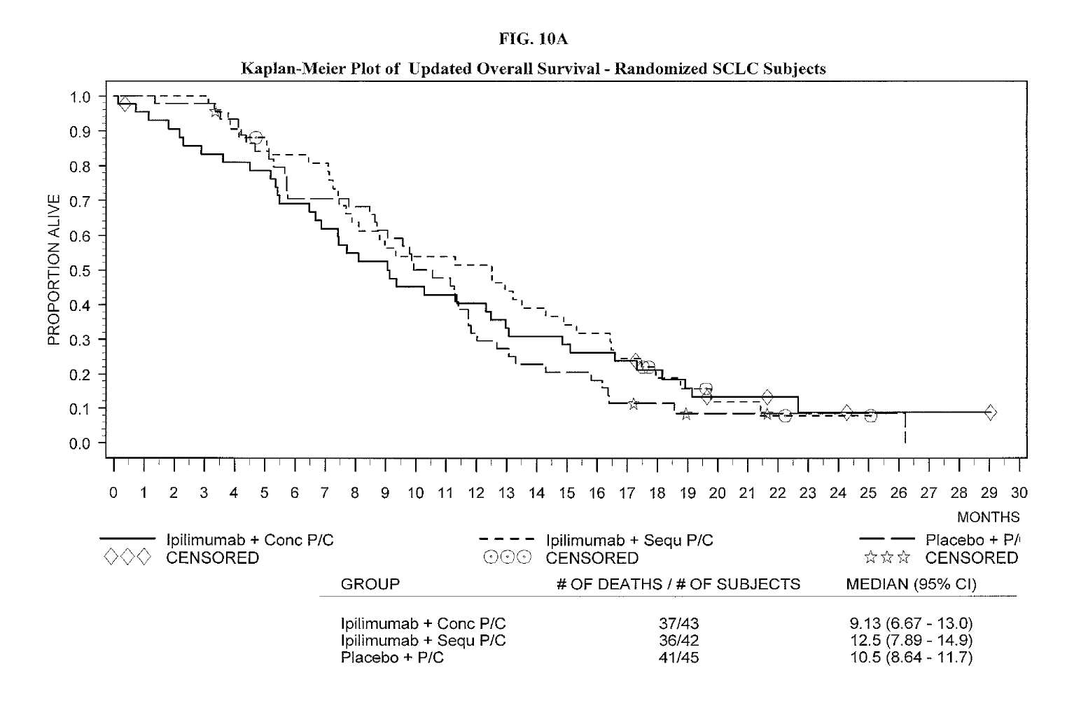

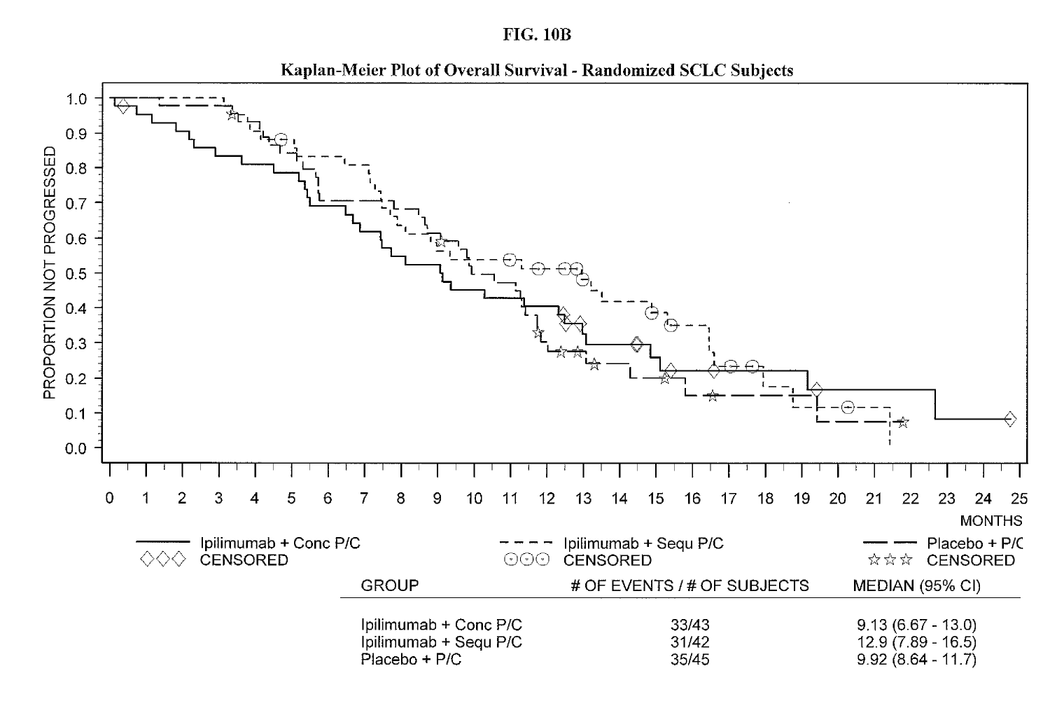

The final results generally supported the prior interim results as shown in Table 2 and FIGS. 10A-B. A total of 130 randomized and 128 treated SCLC subjects were included in the final analysis. A total of 114 subjects have died and 16 subjects were either alive or lost to follow-up at the time of the analysis. The minimum clinical follow-up was 16 months.

The median survival times were 9.1 (95% CI 6.7-13.0), 12.5 (95% CI 7.9-14.9), and 10.5 (95% CI 8.6, 11.7) months for the concurrent arm, phased arm and placebo arm, respectively. (see FIGS. 10A-B).

The hazard ratios are 0.76 (95% CI 0.48, 1.19) and 0.89 (95% CI 0.57-1.39) for the phased arm vs. placebo and concurrent arm v. placebo, respectively.

Numerical improvement in OS was observed in the phased arm which is consistent with the findings from the interim analysis. However, the improvement in median survival time was attenuated.

The study was not powered for overall survival analysis. The Kaplan-Meier curves suggest non-proportional survival hazards.

The sequential/phased arm showed a higher rate of Immune-Related Best Overall Response Rate (irBORR), the highest Best Overall Response Rate using mWHO criteria, the highest Immune-Related Disease Control Rate (irDCR), and the highest Disease Control Rate using mWHO criteria (see FIG. 11).

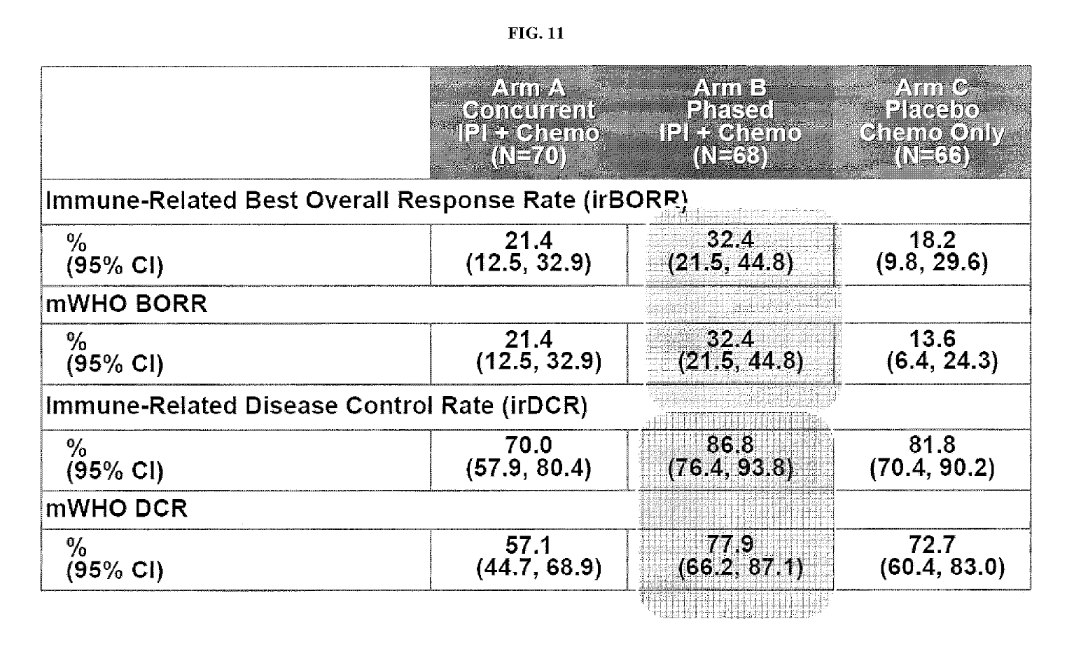

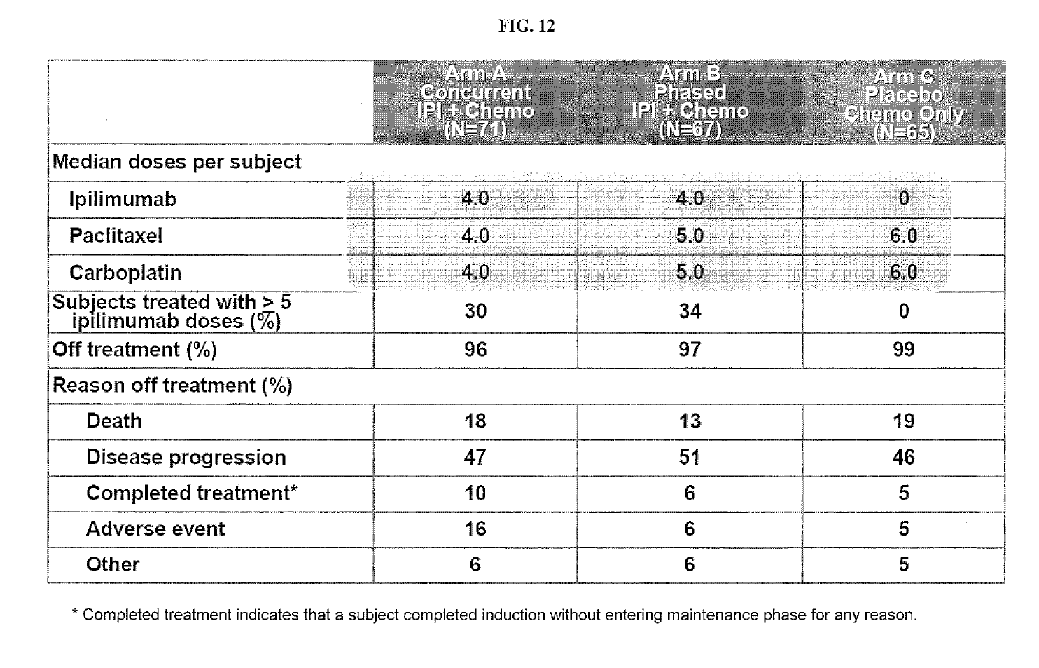

The final discontinuation and disposition were comparable to the results observed for the interim analysis. (see FIG. 12).

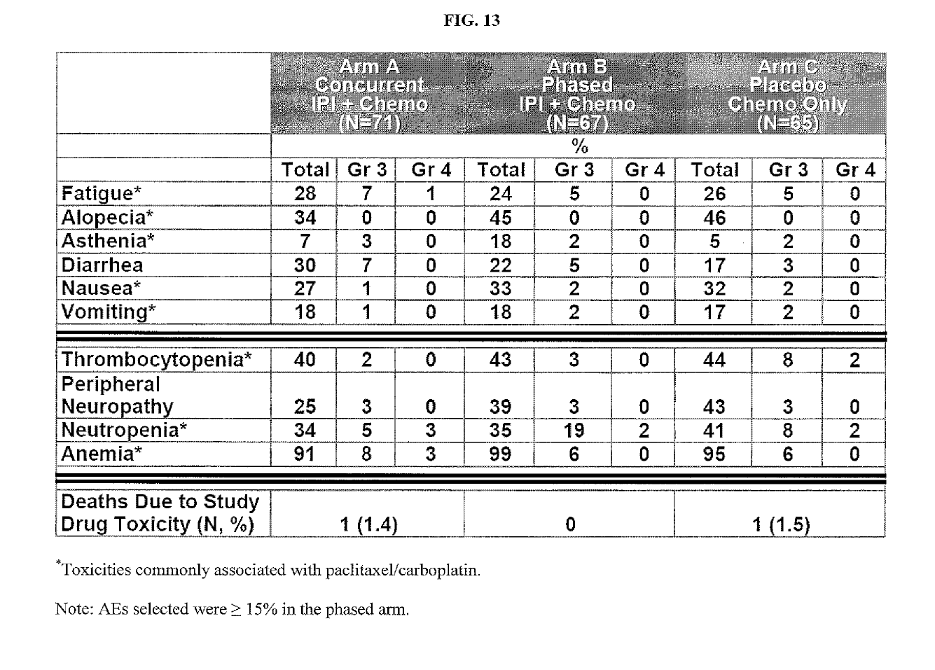

Final analysis of common drug-related adverse events observed a lower level of incidence of grade 3 and grade 4 adverse events for the phased/sequential arm relative to the concurrent arm. (see FIG. 13).

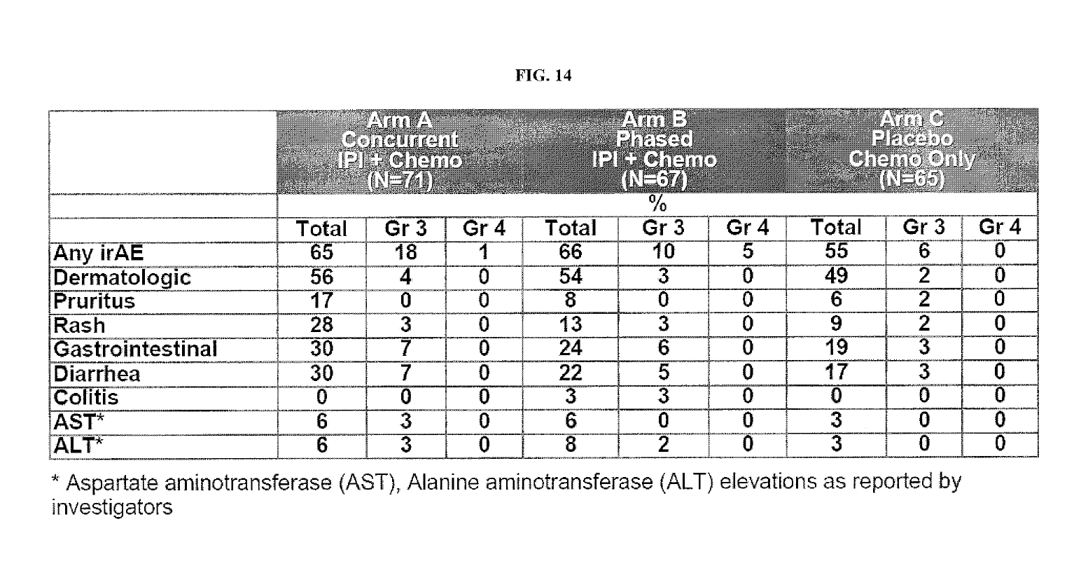

Final analysis of key immune-related adverse events observed a lower level of incidence of grade 3 immune related adverse events, with an elevated rate of grade 4 adverse events, for the phased/sequential arm relative to the concurrent arm. No grade 4 dermatologic or gastrointestinal irAEs were observed. Fatal (grade 5) toxic epidermal necrolysis (TEN) was observed in 1 patient in the concurrent arm, but not in the sequential/phased arm. Hypopituitarism and adrenal insufficiency were not observed. 2 patients experienced grade 1-2 hypothyroidism (1 each in the concurrent arm and the sequential/phased arm, respectively). (see FIG. 14).

TABLE 2
Comparison of Overall Survival Between the Treatment Groups Randomized NSCLC Subjects

| | No. of Events/No. of Subjects | | Log-rank (compare to Arm C) | | Hazard Ratio (compare to Arm C) | |
|---|---|---|---|---|---|---|
| | Median OS(Months) | | p-value | | 95% CI | |
| Treatment | 5 JAN. 2011 | 24 AUG. 2010 | 5 JAN. 2011 | 24 AUG. 2010 | 5 JAN. 2011 | 24 AUG. 2010 |
| Groups | Analysis | Analysis | Analysis | Analysis | Analysis | Analysis |
| Arm A | 37/43 (86.0) | 33/43 (76.7) | — | 0.4132 | 0.888 | 0.947 |
| (concurrent) | 9.13 (6.67, 12.98) | 9.13 (6.67, 12.98) | | | (0.566, 1.392) | (0.585, 1.536) |
| Arm B (phased) | 36/42 (85.7) | 31/42 (73.8) | — | 0.1287 | 0.756 | 0.753 |
| | 12.52 (7.89, 14.88) | 12.94 (7.89, 16.46) | | | (0.479, 1.192) | (0.461, 1.232) |
| Arm C (placebo) | 41/45 (91.1) | 35/45 (77.8) | — | — | — | — |
| | 10.55 (8.64, 11.73) | 9.92 (8.64, 11.73) | | | | |

### DISCUSSION

Greater early progression (within first 6 weeks) observed in concurrent arm may result from Ipilimumab-related tumor inflammation that was considered by the investigators as disease progression while they were tumor flare. Progression whenever assessed, including early progression, was included in the irPFS, PFS, and irDCR analyses.

Numerically greater sequential arm results may have been influenced by an imbalance in ECOG status at baseline.

Overall, the study met its primary endpoint. The final analysis for irPFS in the NSCLC portion of study CA184-041 together with interim analyses for response and survival suggest added effectiveness in the Ipilimumab-containing arms over the carboplatin/paclitaxel control.

All efficacy results must be interpreted with caution given the small sample size and limited power of the study.

A late effect (curves starting to separate after approximately four months for irPFS and six months for OS) might be present in the concurrent schedule but was not readily apparent with the sequential schedule

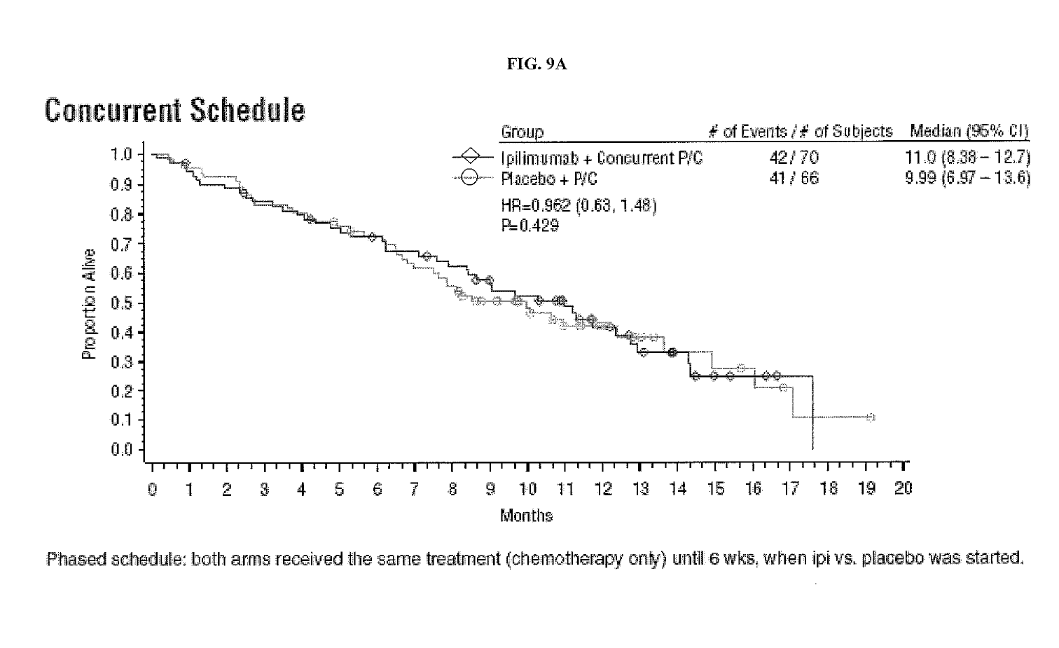

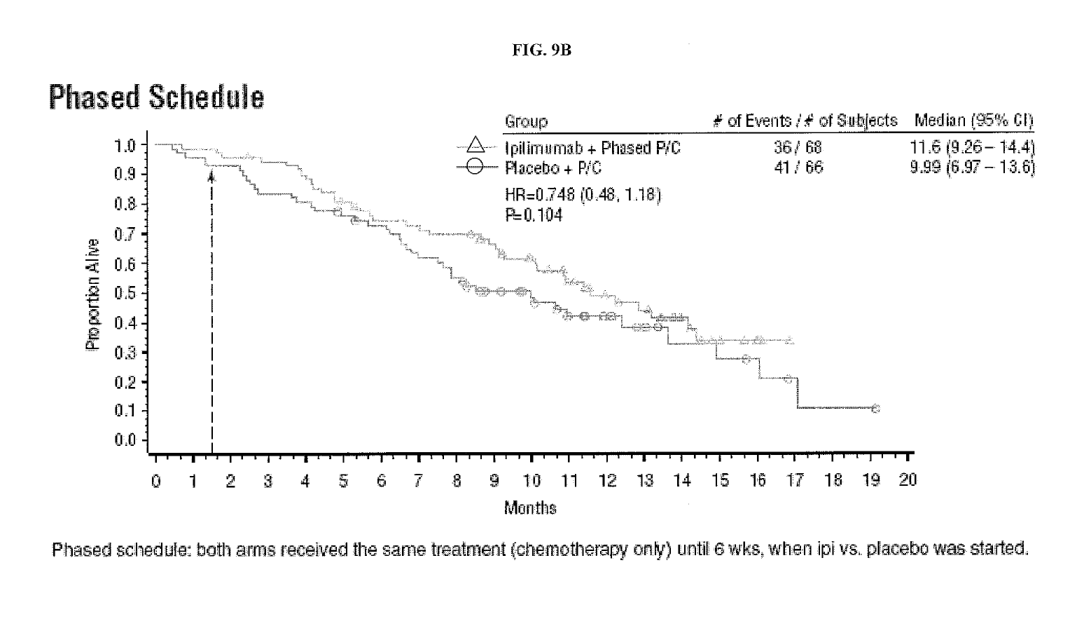

irPFS and OS (interim data) seem to indicate that the sequential schedule might present the best efficacy/safety profile (see FIG. 9).

Final analysis showed Ipilimumab did not potentiate chemotherapy-related toxicity, and that immune-related adverse events were generally manageable with established algorithms. In addition, the final analysis also showed PFS, by mWHO criteria, was extended for the phased schedule only; that a numerical improvement in OS was observed in the phased schedule but was not significant; and BORR was numerically higher when ipilimumab was administered with paclitaxel/carboplatin, with the greater benefit seen in the phased group.

The final data confirm the trend observed during the interim data analysis that the sequential schedule presented the best efficacy/and safety profile (see FIGS. 10A-B, 12, 13, and 14).

The mechanism for why phased administration shows an improvement in efficacy and safety are likely complex and might include: reduction of tumor induced immunosupression reduction of T reg and myeloid suppressor T cell reduction in interstitial pressure. With regards to reduced interstitial pressure, it is known that high interstitial fluid pressure impairs extravasation of macromolecules and cells. Thus, normalization of high Interstitial fluid pressure may facilitate access to the tumor cells for antibodies and effector cells. Additional investigation will be directed at better understanding the mechanism by which phased dosing results in better efficacy and safety for ipilimumab and other immunotherapy-based regimens.

The entire disclosure of each document cited (including patents, patent applications, journal articles, abstracts, laboratory manuals, books, GENBANK® Accession numbers, SWISS-PROT® Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

What is claimed is:

1. A method for treating a patient with cancer comprising the sequential administration of (i) one or more cycles of a chemotherapeutic agent; followed by the administration of (ii) one or more cycles of a combination comprising an immunomodulatory agent and said chemotherapeutic agent.

2. The method of claim 1 wherein said cancer is a solid tumor.

3. The method of claim 2 wherein said cancer is selected from the group consisting of: melanoma, prostate cancer, lung cancer, non-small cell lung cancer, and small cell lung cancer.

4. The method according to claim 1, further comprising an optional Intervening Period in between said one or more cycles of a chemotherapeutic agent and said one or more cycles of said combination.

5. The method of claim 1, 2, 3, or 4 wherein the co-stimulatory pathway modulator is a CTLA-4 antagonist.

6. The method of claim 5 wherein the CTLA-4 antagonist is selected from the group consisting of: Ipilimumab and tremelimumab.

7. The method of claim 1, 2, 3, or 4 wherein the chemotherapeutic agent is selected from the group consisting of: TAXOL®, paclitaxel, carboplatin, a tubulin stabilizing agent, a second co-stimulatory pathway modulator, a taxane, an epothilone, IXEMPRA®, PROVENGE®, Bevacizumab, Dacarbazine, PARAPLATIN®; Budesonide; an inhibitor of CD137; and steroids.

8. The method of claim 6, wherein said co-stimulatory pathway modulator is administered at a dosage of about 0.1 to 15 mg/kg once every three weeks.

9. The method of claim 7, wherein said chemotherapeutic agent is administered in combination with at least one additional chemotherapeutic agent.

10. The method of claim 9, wherein said chemotherapeutic agent combination comprises the additional combination of one or more of the following: TAXOL®, paclitaxel, carboplatin, a tubulin stabilizing agent, a second co-stimulatory pathway modulator, a taxane, an epothilone, IXEMPRA®, PROVENGE®, Bevacizumab, Dacarbazine, PARAPLATIN®; Budesonide; an inhibitor of CD137; and steroids.

11. A method for treating a patient with cancer comprising the sequential administration of (i) one or more cycles of a chemotherapeutic agent; followed by the administration of (ii) one or more cycles of a combination comprising an immunomodulatory agent and said chemotherapeutic agent, wherein said method provides a decreased likelihood the patient will have adverse event(s) relative to a concurrent administration of said agent(s).

12. A method for treating a patient with cancer comprising the sequential administration of (i) one or more cycles of a chemotherapeutic agent; followed by the administration of (ii) one or more cycles a combination comprising an immunomodulatory agent and said chemotherapeutic agent, wherein said method provides a decreased likelihood the patient will discontinue therapy relative to a concurrent administration of said agent(s).

13. A method for treating a patient with cancer comprising the sequential administration of (i) one or more cycles of a chemotherapeutic agent; followed by the administration of (ii) one or more cycles of a combination comprising an anti-CTLA4 antibody.

14. The method according to claim 13 wherein said anti-CTLA4 antibody is Ipilimumab or tremelimumab; and wherein said chemotherapeutic agent comprises an agent selected from the group consisting of: pacitaxel and carboplatin.
